SEQUENCE LISTING (7 sequences)

SEQ ID NO: 1 - protein, 124 aa, Artificial Sequence
  Description of Artificial Sequence/note = synthetic construct
MRIFTFIFAYSQTTFIQLEFSPSSIYYLLLWVKTNLSVSAVCKLAWALRAAGLNRNYPRVNKNVIKFKTTAPSPALDSFMPQMLTVCKCFLCEALAWKHLNLIALYVCLYQKSYAQIHCQPSNS

SEQ ID NO: 2 - DNA, 375 bp, Artificial Sequence
  Description of Artificial Sequence/note = synthetic construct
atgaggatttttacttttattttcgcctacagtcaaactacctttatccaattagaattttcaccctcatctatttattatctcctgctctgggtaaaaacaaacctgagtgtctctgccgtgtgcaaattagcctgggctctgagggcggcagggctgaatagaaactatcctcgagtgaataaaaatgttattaaatttaaaaccacagccccatcaccagcactggattcctttatgccacagatgttgactgtttgcaagtgtttcctctgtgaggcactggcttggaagcatttgaacctaatagcattatatgtgtgtttatatcagaaatcatatgcacagatacattgccagccttcaaattcatag

SEQ ID NO: 3 - DNA, 2090 bp, Artificial Sequence
  Description of Artificial Sequence/note = synthetic construct
ctacctgcactttccttctactgtcacaatatcaatatactcagctacagcttagtctccccatttaacagataaggccactgaggcttagtgagtttcaatgacctgcctgaagtcatggagcagggtgtgacaaaagtgggaaaactggaatcagaatcacttcacccacccctaacctcaccagaacagtgttttcaattctagctacacattaaaatcactggagaggttttaaaaatactgaaacccaggctctgtacacagtgattccatttaactggtctgaggtggggcctaggcactggtatgttttaaaggttttcttggtattttttttttccagttactttctttaaaaaaatttttttttaacttttcatttgaaataatttcagacttccaaaaaaagatgtgaaatagtacaaagcagtcctgtgtatccttcccccggtttttccaaatgttaacatcttacatacccacagcgcagctatcaaagccaggatattaacattgatacaataccattcactaatccacagaccttattcaaacggtatcaatggagcttggtggatagttctaaatcattctggagacctcattatttttagttattccatccctttagtgtacttgaatctttcctaggtgatcttaatgtgcagccagggctgagaactgctgggtgtggacggtggagcctggagttagtattctacgagtagacttaattggctttacggtgcatttttttctccccgttctctgacatgtaagagctatgtctgaaaatctagtatatttctaagctattgttatgctgctgatgggatgaatcatttgaagatttctgactatttttatgaagtgacagttatatctgctgggaggagaaatgagccctagttatattcatagcaaggttactgccacctaggtgaaaaatggaaaatggtgtattggggagggaagagttgaagagctttcaaatatgaactcacaagtcagtgggctaggtcttcgcagaactcaatgccattctttggatgtgagtctggaatacagtttagattttaattagagatgtgctggaccttaatgtaaatgtatttagatagggaagccaaagacactaaactgaaaaccttgcatttcctacagatggtttattaaattgaaatatgtctcttaacctctggtcttgaccctgctgttgcaaagatgagtcattgaaacatcagcctgtctgttatcctgaatgactggctggagtggcccggctggaggcaccacttggtaacacatgaaactggttagttcatgaggatttttacttttattttcgcctacagtcaaactacctttatccaattagaattttcaccctcatctatttattatctcctgctctgggtaaaaacaaacctgagtgtctctgccgtgtgcaaattagcctgggctctgagggcggcagggctgaatagaaactatcctcgagtgaataaaaatgttattaaatttaaaaccacagccccatcaccagcactggattcctttatgccacagatgttgactgtttgcaagtgtttcctctgtgaggcactggcttggaagcatttgaacctaatagcattatatgtgtgtttatatcagaaatcatatgcacagatacattgccagccttcaaattcatagagaaaacaattggctcccgtggtgttaggtagcagccagggctgtggccttgtgggtcccagcacaggagactctttcataaccccctgggctttggaacatgctgctgcctctgccaagtacacccttgtctcccaccacccctcttttcctggctcactgctgccacctcctcttggaagccttccttgatttcctcccccaacctgagttagaagtcttttccatatatttgcacaatactcagtgcttctatgtgctgcagtaaaagtaattgtttatttgtctgatttcttcacatgctagcacctagcccagtgcctgccacatagaaagtgcctatgataatgtgtgtgtgtgaataaatgaatgataaatgaaaaaaaaaa

SEQ ID NO: 4 - DNA, 10 bp, Artificial Sequence
  Description of Artificial Sequence/note = synthetic construct
tctgtgctgg

SEQ ID NO: 5 - DNA, 10 bp, Artificial Sequence
  Description of Artificial Sequence/note = synthetic construct
ccagcacaga

SEQ ID NO: 6 - DNA, 10 bp, Artificial Sequence
  Description of Artificial Sequence/note = synthetic construct
ccagtgctgg

SEQ ID NO: 7 - DNA, 6 bp, Artificial Sequence
  Description of Artificial Sequence/note = synthetic construct
aataaa

(19) United States
(12) Patent Application Publication
(10) Pub. No.: US 2008/0004229 A1
(43) Pub. Date: Jan. 3, 2008

(54) Methods and Compositions Related to Skcg-1, a Tumor Suppressor

(76) Inventor: Roy Deodutta, Miramar, FL (US)

Correspondence Address: NEEDLE & ROSENBERG, P.C., SUITE 1000, 999 PEACHTREE STREET, ATLANTA, GA, 30309-3915, US

(21) Appl. No.: 10/592,995

(22) PCT Filed: Mar. 15, 2005
(86) PCT No.: PCT/US05/08568

§ 371 Date: May 31, 2007

Related U.S. Application Data
(60) Provisional application No. 60/553,419, filed on Mar. 16, 2004.

(51) Int. Cl. A61K 31/7052; A61P 35/00; C07H 21/00; C07K 14/00; C07K 16/30; C12N 1/00; C12N 15/63; C12N 5/00; C12N 5/10; C12Q 1/68

(52) U.S. Cl. 514/44; 435/243; 435/320.1; 435/325; 435/375; 435/6; 530/350; 530/387.3; 530/387.9; 530/391.3; 536/23.5

(57) ABSTRACT

The present invention relates generally to SKCG-1, a novel gene involved in cell growth regulation. The present invention also relates to the detection of SKCG-1 nucleic acids and polypeptides. Methods for administering the nucleic acid and polypeptides of this invention to inhibit cell growth and treat hyperproliferative cell disorders, such as cancer are also provided.

This application claims priority to U.S. provisional application Ser. No. 60/553,419, filed Mar. 16, 2004, which is herein incorporated by this reference in its entirety.

### FIELD OF THE INVENTION

The invention relates generally to SKCG-1, a novel gene involved in cell growth regulation. The present invention also relates to the detection of SKCG-1 nucleic acids and polypeptides. Methods for administering the nucleic acid and polypeptides of this invention to inhibit cell growth and treat hyperproliferative cell disorders, such as cancer, are also provided.

### SUMMARY OF THE INVENTION

The present invention provides a purified SKCG-1 polypeptide comprising SEQ ID NO: 1 or fragments thereof. The polypeptides of the present invention also include polypeptides that comprise a sequence that is at least 95% identical to the sequence SEQ ID NO: 1. Also provided are polypeptides comprising SEQ ID NO: 1 with one or more conservative substitutions. Also provided by the present invention are nucleic acids encoding SKCG-1 polypeptides or fragments thereof.

Also provided by the present invention is a method for detecting expression of a tumor suppressor gene in a subject, comprising analyzing a sample from the subject for expression of a SKCG-1 gene.

Further provided by the present invention is a method for determining whether a subject has or is at risk for developing cancer comprising: detecting reduced expression of the SKCG-1 gene in a test sample from the subject as compared to a control sample, wherein reduced expression of the SKCG-1 gene in the test sample indicates that the subject has or is at risk for developing cancer.

The present invention further provides a method for determining whether a subject has or is at risk for developing cancer comprising: analyzing the methylation status of a SKCG-1 gene in a sample from the subject and detecting a difference in methylation status as compared to that of a comparable normal cell, wherein the difference in methylation status indicates that the subject has or is at risk of developing cancer.

Also provided by the present invention is a method of suppressing growth of a tumor cell, comprising introducing into the tumor cell an expression vector comprising a polynucleotide encoding a polypeptide having the amino acid sequence shown in SEQ ID NO: 1, wherein expression of the polypeptide in the tumor cell, suppresses growth of the tumor cell.

Further provided by the present invention is a method of suppressing tumor cell growth in a subject, comprising introducing into the tumor cell in the subject, an expression vector comprising a polynucleotide encoding a polypeptide having the amino acid sequence of SEQ ID NO: 1, wherein expression of the polypeptide in the tumor cell in the subject, suppresses growth of the tumor cell in the subject.

### BRIEF DESCRIPTION OF THE FIGURES

FIG. 1 shows representative AP-PCR fingerprints of kidney tumor (T) and corresponding uninvolved kidney tissue (N) DNA samples using random ten-mer (5′TCTGTGCTGG3′) (SEQ ID NO: 4) primer. The PCR amplification product was resolved on agarose gel and detected by ethidium bromide staining. The arrow indicates the amplification of 466 bp PCR product with reduced intensity in kidney tumors as compared to their matched normal tissue samples.

FIG. 2 is a sequence chromatogram showing mutation in SKCG-1 in a tumor sample.

FIG. 3 shows the determination of SKCG-1 transcript size by Northern blot analysis. Three concentrations (5 μg, 10 μg, 20 μg) of total RNA isolated from human kidney epithelial cells (HK-2) were resolved on formaldehyde denaturing-agarose gel, transferred on to nylon membrane, and probed with SKCG-1 specific PCR mediated Dig—labeled probe. The same membrane was reprobed with GAPDH probe after stripping of the SKCG-1 probe.

FIG. 4 is a representative photograph of RT-PCR analysis in 4 Wilms tumor samples. Reduction or loss of SKCG-1 transcript in tumor tissue as compared to control tissue was detected. β-actin transcript in N (normal)-T(tumor) pairs confirm the similar amount of RNA taken for RT reaction.

FIG. 5A illustrates FISH analysis performed to confirm that the SKCG-1 BACK clone is located in an area that corresponds to band 11q23.2.

FIG. 5B is an ideogram illustrating the results of FISH analysis showing that the SKCG-1 BAC clone is located at a position, which is 73% of the distance from the centromere to the telomere of chromosome arm 11q, an area that corresponds to band 11q23.2.

FIG. 6 is a photograph of a representative agarose gel showing increased transcript level of SKCG-1 and P⁵³ in HEK-293 cells grown (in triplicate) in serum free media as compared to the cells grown with 10% fetal bovine serum for 72 hrs. Similar level of β-actin transcript in both groups confirm that similar amounts of RNA were taken for the RT reaction.

FIG. 7A is a representative photograph showing siRNA mediated silencing of the SKCG-1 transcript. Total RNA was isolated from HEK293 cells after 72 hrs of siRNA transfection, RT was performed, and transcript level was detected by PCR using SKCG-1 specific primers. Amplification of β-actin was used as control.

FIG. 7B shows the effect of SKCG-1 gene inhibition on the growth of HEK-293 cells. Cells were counted after 72 hrs of siRNA transfection. Gene inhibition was confirmed by RT-PCR analysis.

### DETAILED DESCRIPTION OF THE INVENTION

The present invention provides a novel, previously uncharacterized gene, SKCG-1, that harbors point mutations in malignant tissues like Wilms tumors and RCC (renal cell carcinoma) samples. A full-length cDNA of SKCG-1 of approximately 2.092 kb that encodes an open reading frame of 124 amino acids is provided herein. The presence of a SKCG-1 gene transcript in various human normal tissues and its absence in numerous malignant tissues, including, but not limited to, Wilms, RCC and breast tumor tissues indicate that it can be associated with tumor suppressive activity. Furthermore, the induction of a SKCG-1 transcript by serum-starvation and the increased cell growth observed upon silencing of this gene in HEK293 cells further supports the role of this gene in regulation of cell growth.

Malignant transformation is characterized by alterations in the normal properties of cell growth, adhesion, motility and shape. The multistep nature of this process is now well defined in a number of systems, as well as the fact that genetic changes in specific genes are responsible for both positive and negative contributions to that process. Analysis of the genes involved has identified those which act positively to induce aspects of the transformed state (oncogenes) and more recently, has led to the identification of those which act to block or suppress the malignant phenotype, the tumor-suppressor genes. The importance of these genes in maintaining the normal phenotype has been established, as many human tumors have been shown to lose tumor suppressive function as a consequence of deletion, rearrangement or mutations in tumor supressors such as Rb and p53. The frequent physical or functional loss of these tumor-suppressor genes in specific human malignancies show that these changes contribute to the development of the neoplastic phenotype.

Loss of function of a particular gene may occur by a variety of mechanisms, including the repression of its expression at the RNA level, and a large number of genes whose expression is repressed either in tumors or in cells transformed by positively acting oncogenes, such as v-ras, v-src or SV40 T antigen, have been identified. As mentioned above, the present invention provides a novel, uncharacterized tumor suppressor, SKCG-1, with cell growth inhibiting activity. Also provided are methods for administering the nucleic acid and protein of this invention to inhibit cell growth and treat hyperproliferative cell disorders, such as cancer.

The present invention may be understood more readily by reference to the following detailed description of preferred embodiments of the invention and the Examples included therein and to the Figures and their previous and following description.

Before the present compounds, compositions, articles, devices, and/or methods are disclosed and described, it is to be understood that this invention is not limited to specific nucleic acids, specific polypeptides, or to particular methods, as such may, of course, vary. It is also to be understood that the terminology used herein is for the purpose of describing particular embodiments only and is not intended to be limiting.

As used in the specification and the appended claims, the singular forms “a,” “an” and “the” include plural referents unless the context clearly dictates otherwise. Thus, for example, reference to “an antibody” includes mixtures of antibodies, reference to “a pharmaceutical carrier” includes mixtures of two or more such carriers, and the like.

Ranges may be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint.

“Optional” or “optionally” means that the subsequently described event or circumstance may or may not occur, and that the description includes instances where said event or circumstance occurs and instances where it does not. For example, the phrase “optionally obtained prior to treatment” means obtained before treatment, after treatment, or not at all.

As used throughout, by “subject” is meant an individual. Preferably, the subject is a mammal such as a primate, and, more preferably, a human. The term “subject” includes domesticated animals, such as cats, dogs, etc., livestock (e.g., cattle, horses, pigs, sheep, goats, etc.), and laboratory animals (e.g., mouse, rabbit, rat, gerbil, guinea pig, etc.).

SKCG-1 Polypeptides

The present invention provides an isolated polypeptide comprising SEQ ID NO: 1. SEQ ID NO: 1 is set forth below:

(SEQ ID NO: 1)

MRIFTFIFAYSQTTFIQLEFSPSSIYYLLLWVKTNLSVSAVCKLAWALRA
AGLNRNYPRVNKNVIKFKTTAPSPAIDSFMPQMLTVCKCFLCEALAWKHL
NLIALYVCLYQKSYAQIHCQPSNS

SEQ ID NO: 1 corresponds to a full-length SKCG-1 polypeptide of 124 amino acids. The present invention also provides fragments of SKCG-1 polypeptides, for example, fragments of SEQ ID NO: 1. Fragments of SEQ ID NO: 1 include but are not limited to fragments comprising amino acids 1-10 of SEQ ID NO: 1, fragments comprising amino acids 1-20 of SEQ ID NO: 1, fragments comprising fragments comprising amino acids 1-30 of SEQ ID NO: , fragments comprising amino acids 1-40 of SEQ ID NO: 1, fragments comprising amino acids 1-50 of SEQ ID NO: 1, fragments comprising amino acids 115-125 of SEQ ID NO: 1, fragments comprising amino acids 100-115 of SEQ ID NO: 1, fragments comprising amino acids 90-100 of SEQ ID NO: 1, fragments comprising amino acids 80-90 of SEQ ID NO: 1 etc. These examples are not meant to be limiting as any fragment comprising at least 5, 10, 15, 20, 25, 30, 35, 40, 45, 50, 55, 60, 65, 70, 75, 80, 85, 90, 95, 100, 105, 110, 115, 120 or 123 contiguous amino acids of SEQ ID NO: 1 is contemplated by this invention. These fragments can be of sufficient length to serve as antigenic peptides for the generation of anti-SKCG-1 antibodies. The present invention also contemplates functional fragments of SKCG-1 that possess at least one activity of SKCG-1, such as, tumor suppressive activity or cell growth inhibiting activity. Fragments and variants of SKCG-1 can include one or more conservative amino acid residues as compared to the amino acid sequence of SEQ ID NO: 1.

By “isolated polypeptide” or “purified polypeptide” is meant a polypeptide that is substantially free from the materials with which the polypeptide is normally associated in nature or in culture. The polypeptides of the invention can be obtained, for example, by extraction from a natural source if available (for example, a mammalian cell), by expression of a recombinant nucleic acid encoding the polypeptide (for example, in a cell or in a cell-free translation system), or by chemically synthesizing the polypeptide. In addition, a polypeptide may be obtained by cleaving full length polypeptides. When the polypeptide is a fragment of a larger naturally occurring polypeptide, the isolated polypeptide is shorter than and excludes the full-length, naturally-occurring polypeptide of which it is a fragment.

The polypeptides of the invention can be prepared using any of a number of chemical polypeptide synthesis techniques well known to those of ordinary skill in the art including solution methods and solid phase methods. One method of producing the polypeptides of the present invention is to link two or more peptides or polypeptides together by protein chemistry techniques. For example, peptides or polypeptides can be chemically synthesized using currently available laboratory equipment using either Fmoc (9-fluorenylmethyloxycarbonyl) or Boc (tert-butyloxycarbonoyl) chemistry. (Applied Biosystems, Inc., Foster City, Calif.). One skilled in the art can readily appreciate that a peptide or polypeptide corresponding to the antibody of the present invention, for example, can be synthesized by standard chemical reactions. For example, a peptide or polypeptide can be synthesized and not cleaved from its synthesis resin, whereas the other fragment of an antibody can be synthesized and subsequently cleaved from the resin, thereby exposing a terminal group which is functionally blocked on the other fragment. By peptide condensation reactions, these two fragments can be covalently joined via a peptide bond at their carboxyl and amino termini, respectively, to form an antibody, or fragment thereof. (Grant GA (1992) Synthetic Peptides: A User Guide. W.H. Freeman and Co., N.Y. (1992); Bodansky M and Trost B., Ed. (1993) Principles of Peptide Synthesis. Springer-Verlag Inc., NY. Alternatively, the peptide or polypeptide is independently synthesized in vivo as described above. Once isolated, these independent peptides or polypeptides may be linked to form an antibody or fragment thereof via similar peptide condensation reactions.

For example, enzymatic ligation of cloned or synthetic peptide segments allow relatively short peptide fragments to be joined to produce larger peptide fragments, polypeptides or whole protein domains (Abrahmsen L et al., Biochemistry, 30:4151 (1991)). Alternatively, native chemical ligation of synthetic peptides can be utilized to synthetically construct large peptides or polypeptides from shorter peptide fragments. This method consists of a two-step chemical reaction (Dawson et al. Synthesis of Proteins by Native Chemical Ligation. Science, 266:776-779 (1994)). The first step is the chemoselective reaction of an unprotected synthetic peptide-alpha-thioester with another unprotected peptide segment containing an amino-terminal Cys residue to give a thioester-linked intermediate as the initial covalent product. Without a change in the reaction conditions, this intermediate undergoes spontaneous, rapid intramolecular reaction to form a native peptide bond at the ligation site. Application of this native chemical ligation method to the total synthesis of a protein molecule is illustrated by the preparation of human interleukin 8 (IL-8) (Baggiolini M et al. (1992) FEBS Lett. 307:97-101; Clark-Lewis I et al., J. Biol. Chem., 269:16075 (1994); Clark-Lewis I et al., Biochemistry, 30:3128 (1991); Rajarathnam K et al., Biochemistry 33:6623-30 (1994)).

Alternatively, unprotected peptide segments are chemically linked where the bond formed between the peptide segments as a result of the chemical ligation is an unnatural (non-peptide) bond (Schnolzer, M et al. Science, 256:221 (1992)). This technique has been used to synthesize analogs of protein domains as well as large amounts of relatively pure proteins with full biological activity (deLisle Milton R C et al., Techniques in Protein Chemistry IV. Academic Press, New York, pp. 257-267 (1992)).

The polypeptides of the invention can also be prepared by other means including, for example, recombinant techniques. Examples of appropriate cloning and sequencing techniques, and instructions sufficient to direct persons of skill through many cloning exercises are found in Sambrook et al. (2001) Molecular Cloning—A Laboratory Manual (3rd ed.) Vol. 1-3, Cold Spring Harbor Laboratory, Cold Spring Harbor Press, NY, (Sambrook).

Also provided by the present invention is a polypeptide comprising an amino acid sequence at least about 70%, 75%, 80%, 85%, 90%, 95%, 96%, 97%, 98% or 99% identical to SEQ ID NO: 1 or fragments of SEQ ID NO: 1.

It is understood that as discussed herein the use of the terms “homology” and “identity” mean the same thing as similarity. Thus, for example, if the use of the word homology is used to refer to two non-natural sequences, it is understood that this is not necessarily indicating an evolutionary relationship between these two sequences, but rather is looking at the similarity or relatedness between their nucleic acid sequences. Many of the methods for determining homology between two evolutionarily related molecules are routinely applied to any two or more nucleic acids or proteins for the purpose of measuring sequence similarity regardless of whether they are evolutionarily related.

In general, it is understood that one way to define any known variants and derivatives or those that might arise, of the disclosed nucleic acids and polypeptides herein, is through defining the variants and derivatives in terms of homology to specific known sequences. In general, variants of nucleic acids and polypeptides herein disclosed typically have at least, about 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99 percent homology to the stated sequence or the native sequence. Those of skill in the art readily understand how to determine the homology of two polypeptides or nucleic acids. For example, the homology can be calculated after aligning the two sequences so that the homology is at its highest level.

Another way of calculating homology can be performed by published algorithms. Optimal alignment of sequences for comparison may be conducted by the local homology algorithm of Smith and Waterman Adv. Appl. A Math. 2: 482 (1981), by the homology alignment algorithm of Needleman and Wunsch, J. Mol. Biol. 48: 443 (1970), by the search for similarity method of Pearson and Lipman, Proc. Natl. Acad. Sci. U.S.A. 85: 2444 (1988), by computerized implementations of these algorithms (GAP, BESTFIT, FASTA, and TFASTA in the Wisconsin Genetics Software Package, Genetics Computer Group, 575 Science Dr., Madison, Wis.; the BLAST algorithm of Tatusova and Madden FEMS Microbiol. Lett. 174: 247-250 (1999) available from the National Center for Biotechnology Information (http://www.ncbi.nlm.nih.gov/blast/b112seq/b12.html)), or by inspection.

The same types of homology can be obtained for nucleic acids by for example the algorithms disclosed in Zuker, M. Science 244:48-52, 1989, Jaeger et al. Proc. Natl. Acad. Sci. USA 86:7706-7710, 1989, Jaeger et al. Methods Enzymol. 183:281-306, 1989 which are herein incorporated by reference for at least material related to nucleic acid alignment. It is understood that any of the methods typically can be used and that in certain instances the results of these various methods may differ, but the skilled artisan understands if identity is found with at least one of these methods, the sequences would be said to have the stated identity.

For example, as used herein, a sequence recited as having a particular percent homology to another sequence refers to sequences that have the recited homology as calculated by any one or more of the calculation methods described above. For example, a first sequence has 80 percent homology, as defined herein, to a second sequence if the first sequence is calculated to have 80 percent homology to the second sequence using the Zuker calculation method even if the first sequence does not have 80 percent homology to the second sequence as calculated by any of the other calculation methods. As another example, a first sequence has 80 percent homology, as defined herein, to a second sequence if the first sequence is calculated to have 80 percent homology to the second sequence using both the Zuker calculation method and the Pearson and Lipman calculation method even if the first sequence does not have 80 percent homology to the second sequence as calculated by the Smith and Waterman calculation method, the Needleman and Wunsch calculation method, the Jaeger calculation methods, or any of the other calculation methods. As yet another example, a first sequence has 80 percent homology, as defined herein, to a second sequence if the first sequence is calculated to have 80 percent homology to the second sequence using each of calculation methods (although, in practice, the different calculation methods will often result in different calculated homology percentages).

Also provided by the present invention is a polypeptide comprising SEQ ID NO: 1, with one or more conservative amino acid substitutions. These, conservative substitutions are such that a naturally occurring amino acid is replaced by one having similar properties. Such conservative substitutions do not alter the function of the polypeptide. For example, conservative substitutions can be made according to the following table:

TABLE 1
Amino Acid Substitutions

| Original Residue | Exemplary Substitutions |
|---|---|
| Arg | Lys |
| Asn | Gln |
| Asp | Glu |
| Cys | Ser |
| Gln | Asn |
| Glu | Asp |
| Gly | Pro |
| His | Gln |
| Ile | leu; val |
| Leu | ile; val |
| Lys | arg; gln |
| Met | leu; ile |
| Phe | met; leu; tyr |
| Ser | Thr |
| Thr | Ser |
| Trp | Tyr |
| Tyr | trp; phe |
| Val | ile; leu |

Thus, it is understood that, where desired, modifications and changes may be made in the nucleic acid encoding the polypeptides of this invention and/or amino acid sequence of the polypeptides of the present invention and still obtain a polypeptide having like or otherwise desirable characteristics. Such changes may occur in natural isolates or may be synthetically introduced using site-specific mutagenesis, the procedures for which, such as mis-match polymerase chain reaction (PCR), are well known in the art. For example, certain amino acids may be substituted for other amino acids in a polypeptide without appreciable loss of functional activity. It is thus contemplated that various changes may be made in the amino acid sequence of the polypeptides of the present invention (or underlying nucleic acid sequence) without appreciable loss of biological utility or activity and possibly with an increase in such utility or activity.

Nucleic Acids

The present invention also provides a nucleic acids that encode SKCG-1 polypeptides and variants or fragments thereof. The present invention also provides a nucleic acid encoding a polypeptide comprising SEQ ID NO: 1 or fragments thereof.

Examples of nucleic acids encoding a polypeptide comprising SEQ ID NO: 1 are provided herein as SEQ ID NO: 2 and SEQ ID NO: 3. SEQ ID NO: 3 is a full-length SKCG-1 cDNA that encodes SEQ ID NO: 1. Furthermore, SEQ ID NO: 3 comprises SEQ ID NO: 2, which is the portion of the SKCG-1 cDNA encoding the SKCG-1 open reading frame of 124 amino acids.

As used herein, the term “nucleic acid” refers to single or multiple stranded molecules which may be DNA or RNA, or any combination thereof, including modifications to those nucleic acids. The nucleic acid may represent a coding strand or its complement, or any combination thereof. Nucleic acids may be identical in sequence to the sequences which are naturally occurring for any of the moieties discussed herein or may include alternative codons which encode the same amino acid as that which is found in the naturally occurring sequence. These nucleic acids can also be modified from their typical structure. Such modifications include, but are not limited to, methylated nucleic acids, the substitution of a non-bridging oxygen on the phosphate residue with either a sulfur (yielding phosphorothioate deoxynucleotides), selenium (yielding phosphorselenoate deoxynucleotides), or methyl groups (yielding methylphosphonate deoxynucleotides), a reduction in the AT content of AT rich regions, or replacement of non-preferred codon usage of the expression system to preferred codon usage of the expression system. The nucleic acid can be directly cloned into an appropriate vector, or if desired, can be modified to facilitate the subsequent cloning steps. Such modification steps are routine, an example of which is the addition of oligonucleotide linkers which contain restriction sites to the termini of the nucleic acid. General methods are set forth in in Sambrook et al. (2001) Molecular Cloning—A Laboratory Manual (3rd ed.) Vol. 1-3, Cold Spring Harbor Laboratory, Cold Spring Harbor Press, NY, (Sambrook).

Once the nucleic acid sequence is obtained, the sequence encoding the specific amino acids can be modified or changed at any particular amino acid position by techniques well known in the art. For example, PCR primers can be designed which span the amino acid position or positions and which can substitute any amino acid for another amino acid. Alternatively, one skilled in the art can introduce specific mutations at any point in a particular nucleic acid sequence through techniques for point mutagenesis. General methods are set forth in Smith, M. “In vitro mutagenesis” Ann. Rev. Gen., 19:423-462 (1985) and Zoller, M. J. “New molecular biology methods for protein engineering” Curr. Opin. Struct. Biol., 1:605-610 (1991), which are incorporated herein in therein entirety for the methods. These techniques can be used to alter the coding sequence without altering the amino acid sequence that is encoded.

Vectors, Cells, and Methods of Using

Also provided is a vector, comprising a nucleic acid of the present invention. The vector can direct the in vivo or in vitro synthesis of any of the polypeptides described herein. The vector is contemplated to have the necessary functional elements that direct and regulate transcription of the inserted nucleic acid. These functional elements include, but are not limited to, a promoter, regions upstream or downstream of the promoter, such as enhancers that may regulate the transcriptional activity of the promoter, an origin of replication, appropriate restriction sites to facilitate cloning of inserts adjacent to the promoter, antibiotic resistance genes or other markers which can serve to select for cells containing the vector or the vector containing the insert, RNA splice junctions, a transcription termination region, or any other region which may serve to facilitate the expression of the inserted gene or hybrid gene. (See generally, Sambrook et al.). The vector, for example, can be a plasmid. The vectors can contain genes conferring hygromycin resistance, gentamicin resistance, or other genes or phenotypes suitable for use as selectable markers, or methotrexate resistance for gene amplification. The vector can comprise the nucleic acid in pET15b, pSRα-Neo, pPICZα, or pPIC9K.

There are numerous other E. coli (Escherichia coli) expression vectors, known to one of ordinary skill in the art, which are useful for the expression of the nucleic acid insert. Other microbial hosts suitable for use include bacilli, such as Bacillus subtilis, and other enterobacteriaceae, such as Salmonella, Serratia, and various Pseudomonas species. In these prokaryotic hosts one can also make expression vectors, which will typically contain expression control sequences compatible with the host cell (e.g., an origin of replication). In addition, any number of a variety of well-known promoters will be present, such as the lactose promoter system, a tryptophan (Trp) promoter system, a beta-lactamase promoter system, or a promoter system from phage lambda. The promoters will typically control expression, optionally with an operator sequence, and have ribosome binding site sequences for example, for initiating and completing transcription and translation. If necessary, an amino terminal methionine can be provided by insertion of a Met codon 5′ and in-frame with the downstream nucleic acid insert. Also, the carboxy-terminal extension of the nucleic acid insert can be removed using standard oligonucleotide mutagenesis procedures. Also, nucleic acid modifications can be made to promote amino terminal homogeneity.

Additionally, yeast expression can be used. The invention provides a nucleic acid encoding a polypeptide of the present invention, wherein the nucleic acid can be expressed by a yeast cell. More specifically, the nucleic acid can be expressed by Pichia pastoris or S. cerevisiae. There are several advantages to yeast expression systems, which include, for example, Saccharoinyces cerevisiae and Pichia pastoris. First, evidence exists that proteins produced in a yeast secretion systems exhibit correct disulfide pairing. Second, efficient large scale production can be carried out using yeast expression systems. The Saccharomyces cerevisiae pre-pro-alpha mating factor leader region (encoded by the MFα-1 gene) can be used to direct protein secretion from yeast (Brake, et al.). The leader region of pre-pro-alpha mating factor contains a signal peptide and a pro-segment which includes a recognition sequence for a yeast protease encoded by the KEX2 gene: this enzyme cleaves the precursor protein on the carboxyl side of a Lys-Arg dipeptide cleavage signal sequence. The nucleic acid coding sequence can be fused in-frame to the pre-pro-alpha mating factor leader region. This construct can be put under the control of a strong transcription promoter, such as the alcohol dehydrogenase I promoter, alcohol oxidase I promoter, a glycolytic promoter, or a promoter for the galactose utilization pathway. The nucleic acid coding sequence is followed by a translation termination codon which is followed by transcription termination signals. Alternatively, the nucleic acid coding sequences can be fused to a second protein coding sequence, such as Sj26 or beta-galactosidase, used to facilitate purification of the fusion protein by affinity chromatography. The insertion of protease cleavage sites to separate the components of the fusion protein is applicable to constructs used for expression in yeast. Efficient post translational glycosylation and expression of recombinant proteins can also be achieved in Baculovirus systems.

Mammalian cells permit the expression of proteins in an environment that favors important post-translational modifications such as folding and cysteine pairing, addition of complex carbohydrate structures, and secretion of active protein. Vectors useful for the expression of active proteins in mammalian cells are characterized by insertion of the protein coding sequence between a strong viral promoter and a polyadenylation signal. The vectors can contain genes conferring hygromycin resistance, genticin or G418 resistance, or other genes or phenotypes suitable for use as selectable markers, or methotrexate resistance for gene amplification. The chimeric protein coding sequence can be introduced into a Chinese hamster ovary (CHO) cell line using a methotrexate resistance-encoding vector, or other cell lines using suitable selection markers. Presence of the vector DNA in transformed cells can be confirmed by Southern blot analysis. Production of RNA corresponding to the insert coding sequence can be confirmed by Northern blot analysis. A number of other suitable host cell lines capable of secreting intact human proteins have been developed in the art, and include the CHO cell lines, HeLa cells, myeloma cell lines, Jurkat cells, etc. Expression vectors for these cells can include expression control sequences, such as an origin of replication, a promoter, an enhancer, and necessary information processing sites, such as ribosome binding sites, RNA splice sites, polyadenylation sites, and transcriptional terminator sequences. Preferred expression control sequences are promoters derived from immunoglobulin genes, SV40, Adenovirus, Bovine Papilloma Virus, etc. The vectors containing the nucleic acid segments of interest can be transferred into the host cell by well-known methods, which vary depending on the type of cellular host. For example, calcium chloride transformation is commonly utilized for prokaryotic cells, whereas calcium phosphate, DEAE dextran, or lipofectin mediated transfection or electroporation may be used for other eukaryotic cellular hosts.

Alternative vectors for the expression of genes or nucleic acids in mammalian cells, those similar to those developed for the expression of human gamma-interferon, tissue plasminogen activator, clotting Factor VIII, hepatitis B virus surface antigen, protease Nexinl, and eosinophil major basic protein, can be employed. Further, the vector can include CMV promoter sequences and a polyadenylation signal available for expression of inserted nucleic acids in mammalian cells (such as COS-7).

Insect cells also permit the expression of mammalian proteins. Recombinant proteins produced in insect cells with baculovirus vectors undergo post-translational modifications similar to that of wild-type proteins. Briefly, baculovirus vectors useful for the expression of active proteins in insect cells are characterized by insertion of the protein coding sequence downstream of the Autographica californica nuclear polyhedrosis virus (AcNPV) promoter for the gene encoding polyhedrin, the major occlusion protein. Cultured insect cells such as Spodoptera frugiperda cell lines are transfected with a mixture of viral and plasmid DNAs and the viral progeny are plated. Deletion or insertional inactivation of the polyhedrin gene results in the production of occlusion negative viruses which form plaques that are distinctively different from those of wild-type occlusion positive viruses. These distinctive plaque morphologies allow visual screening for recombinant viruses in which the AcNPV gene has been replaced with a hybrid gene of choice.

The invention also provides for the vectors containing the contemplated nucleic acids in a host suitable for expressing the nucleic acids. The host cell can be a prokaryotic cell, including, for example, a bacterial cell. More particularly, the bacterial cell can be an E. coli cell. Alternatively, the cell can be a eukaryotic cell, including, for example, a Chinese hamster ovary (CHO) cell, a myeloma cell, a Pichia cell, or an insect cell. The coding sequence for any of the polypeptides described herein can be introduced into a Chinese hamster ovary (CHO) cell line, for example, using a methotrexate resistance-encoding vector, or other cell lines using suitable selection markers. Presence of the vector DNA in transformed cells can be confirmed by Southern blot analysis. Production of RNA corresponding to the insert coding sequence can be confirmed by Northern blot analysis. A number of other suitable host cell lines have been developed and include myeloma cell lines, fibroblast cell lines, and a variety of tumor cell lines such as melanoma cell lines. Expression vectors for these cells can include expression control sequences, such as an origin of replication, a promoter, an enhancer, and necessary information processing sites, such as ribosome binding sites, RNA splice sites, polyadenylation sites, and transcriptional terminator sequences. Preferred expression control sequences are promoters derived from immunoglobulin genes, SV40, Adenovirus, Bovine Papilloma Virus, etc. The vectors containing the nucleic acid segments of interest can be transferred into the host cell by well-known methods, which vary depending on the type of cellular host. For example, calcium chloride transformation is commonly utilized for prokaryotic cells, whereas calcium phosphate, DEAE dextran, or lipofectin mediated transfection or electroporation may be used for other cellular hosts.

The present invention provides a method of making any of the SKCG-1 polypeptides, fragments and variants described herein comprising: culturing a host cell comprising a vector that encodes a SKCG-1 polypeptide and purifying the polypeptide produced by the host cell. As mentioned above, these polypeptides include, but are not limited to, a polypeptide comprising SEQ ID NO: 1 or a fragment thereof, polypeptides comprising an amino acid sequence at least about 95% identical to the sequence of SEQ ID NO: 1 or a fragment thereof and polypeptides comprising the amino acid sequence of SEQ ID NO: 1, or a fragment thereof, with one or more conservative amino acid substitutions.

Antibodies

The present invention provides an isolated antibody or fragment thereof that specificatlly binds a SKCG-1 polypeptide or a fragment thereof. The present invention further provides an isolated antibody or fragment thereof that specifically binds an epitope contained within amino acids 1-124 of the SKCG-1 polypeptide. In other words, the present invention provides an isolated antibody or fragment thereof that specifically binds an epitope contained within the amino acid sequence of SEQ ID NO: 1. The present invention also provides an antibody that specifically binds to an epitope contained within amino acids 1-124 of the SKCG-1 polypeptide, as evidenced by competitive binding studies. Competitive binding studies are well known in the art. For example, if a test antibody competes for binding to the SKCG-1 polypeptide with an antibody or ligand that specifically binds an epitope contained within amino acids 1-124 of the SKCG-1 polypeptide, one of skill in the art would readily know that the test antibody binds the same epitope as the antibody or ligand that specifically binds to an epitope contained within or overlapping with amino acids 1-124 of the SKCG-1 polypeptide.

The antibody of the present invention can be a polyclonal antibody or a monoclonal antibody. The antibody of the invention selectively binds a SKCG-1 polypeptide. By “selectively binds” or “specifically binds” is meant an antibody binding reaction which is determinative of the presence of the antigen (in the present case, a SKCG-1 polypeptide or antigenic fragments thereof among a heterogeneous population of proteins and other biologics. Thus, under designated immunoassay conditions, the specified antibodies bind preferentially to a particular peptide and do not bind in a significant amount to other proteins in the sample. Specific binding to a SKCG-1 polypeptide under such conditions requires an antibody that is selected for its specificity to a SKCG-1 polypeptide. Preferably, selective binding includes binding at about or above 1.5 times assay background and the absence of significant binding is less than 1.5 times assay background.

This invention also contemplates antibodies that compete for binding to natural SKCG-1 interactors. For example, an antibody of the present invention can compete with SKCG-1 for a binding site (e.g. a receptor) on a cell or the antibody can compete with SKCG-1 for binding to another protein or biological molecule, such as a nucleic acid that is under the transcriptional control of SKCG-1. The antibody optionally can have either an antagonistic or agonistic function as compared to the antigen.

Preferably, the antibody binds a SKCG-1 polypeptide ex vivo or in vivo. Optionally, the antibody of the invention is labeled with a detectable moiety. For example, the detectable moiety can be selected from the group consisting of a fluorescent moiety, an enzyme-linked moiety, a biotin moiety and a radiolabeled moiety. The antibody can be used in techniques or procedures such as diagnostics, screening, or imaging. Anti-idiotypic antibodies and affinity matured antibodies are also considered to be part of the invention.

As used herein, the term “antibody” encompasses, but is not limited to, whole immunoglobulin (i.e., an intact antibody) of any class. Native antibodies are usually heterotetrameric glycoproteins, composed of two identical light (L) chains and two identical heavy (H) chains. Typically, each light chain is linked to a heavy chain by one covalent disulfide bond, while the number of disulfide linkages varies between the heavy chains of different immunoglobulin isotypes. Each heavy and light chain also has regularly spaced intrachain disulfide bridges. Each heavy chain has at one end a variable domain (V(H)) followed by a number of constant domains. Each light chain has a variable domain at one end (V(L)) and a constant domain at its other end; the constant domain of the light chain is aligned with the first constant domain of the heavy chain, and the light chain variable domain is aligned with the variable domain of the heavy chain. Particular amino acid residues are believed to form an interface between the light and heavy chain variable domains. The light chains of antibodies from any vertebrate species can be assigned to one of two clearly distinct types, called kappa (k) and lambda (l), based on the amino acid sequences of their constant domains. Depending on the amino acid sequence of the constant domain of their heavy chains, immunoglobulins can be assigned to different classes. There are five major classes of human immunoglobulins: IgA, IgD, IgE, IgG and IgM, and several of these may be further divided into subclasses (isotypes), e.g., IgG-1, IgG-2, IgG-3, and IgG-4; IgA-1 and IgA-2. One skilled in the art would recognize the comparable classes for mouse or other species. The heavy chain constant domains that correspond to the different classes of immunoglobulins are called alpha, delta, epsilon, gamma, and mu, respectively.

As used herein, the terms “immunoglobulin heavy chain or fragments thereof” and “immunoglobulin light chain or fragments thereof” encompass chimeric peptides and hybrid peptides, with dual or multiple antigen or epitope specificities, and fragments, including hybrid fragments. Thus, fragments of the heavy chains and/or fragments of the light chains that retain the ability to bind their specific antigens are provided. For example, fragments of the heavy chains and/or fragments of the light chains that maintain SKCG-1 protein binding activity are included within the meaning of the terms “immunoglobulin heavy chain or fragments thereof” and “immunoglobulin light chain and fragments thereof,” respectively. Such heavy chains and light chains and fragments thereof, respectively, can be made by techniques known in the art and can be screened for specificity and activity according to the methods set forth in the Examples and in general methods for producing antibodies and screening antibodies for specificity and activity (See Harlow and Lane. Antibodies, A Laboratory Manual. Cold Spring Harbor Publications, New York, (1988)).

The term “variable” is used herein to describe certain portions of the variable domains that differ in sequence among antibodies and are used in the binding and specificity of each particular antibody for its particular antigen. However, the variability is not usually evenly distributed through the variable domains of antibodies. It is typically concentrated in three segments called complementarity determining regions (CDRs) or hypervariable regions both in the light chain and the heavy chain variable domains. The more highly conserved portions of the variable domains are called the framework (FR). The variable domains of native heavy and light chains each comprise four FR regions, largely adopting a β-sheet configuration, connected by three CDRs, which form loops connecting, and in some cases forming part of, the β-sheet structure. The CDRs in each chain are held together in close proximity by the FR regions and, with the CDRs from the other chain, contribute to the formation of the antigen binding site of antibodies (see Kabat E. A. et al., “Sequences of Proteins of Immunological Interest,” National Institutes of Health, Bethesda, Md. (1987)). The constant domains are not involved directly in binding an antibody to an antigen, but exhibit various effector functions, such as participation of the antibody in antibody-dependent cellular toxicity.

As used herein, the term “antibody or fragments thereof” encompasses chimeric antibodies and hybrid antibodies, with dual or multiple antigen or epitope specificities, and fragments, such as F(ab′)2, Fab′, Fab and the like, including hybrid fragments. Thus, fragments of the antibodies that retain the ability to bind their specific antigens are provided. For example, fragments of antibodies which maintain SKCG-1 protein binding activity are included within the meaning of the term “antibody or fragment thereof.” Such antibodies and fragments can be made by techniques known in the art and can be screened for specificity and activity according to the methods set forth in the Examples and in general methods for producing antibodies and screening antibodies for specificity and activity (See Harlow and Lane. Antibodies, A Laboratory Manual. Cold Spring Harbor Publications, New York, (1988)).

Also included within the meaning of “antibody or fragments thereof” are conjugates of antibody fragments and antigen binding proteins (single chain antibodies) as described, for example, in U.S. Pat. No. 4,704,692, the contents of which are hereby incorporated by reference.

Optionally, the antibodies are generated in other species and “humanized” for administration in humans. In one embodiment of the invention, the “humanized” antibody is a human version of the antibody produced by a germ line mutant animal. Humanized forms of non-human (e.g., murine) antibodies are chimeric immunoglobulins, immunoglobulin chains or fragments thereof (such as Fv, Fab, Fab′, F(ab′)2, or other antigen-binding subsequences of antibodies) which contain minimal sequence derived from non-human immunoglobulin. Humanized antibodies include human immunoglobulins (recipient antibody) in which residues from a CDR of the recipient are replaced by residues from a CDR of a non-human species (donor antibody) such as mouse, rat or rabbit having the desired specificity, affinity and capacity. In one embodiment, the present invention provides a humanized version of an antibody, comprising at least one, two, three, four, or up to all CDRs of a SKCG-1 monoclonal antibody. In some instances, Fv framework residues of the human immunoglobulin are replaced by corresponding non-human residues. Humanized antibodies may also comprise residues that are found neither in the recipient antibody nor in the imported CDR or framework sequences. In general, the humanized antibody will comprise substantially all of or at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region (Fc), typically that of a human immunoglobulin (Jones et al., Nature, 321:522-525 (1986); Riechmann et al., Nature, 332:323-327 (1988); and Presta, Curr. Op. Struct. Biol., 2:593-596 (1992)).

Methods for humanizing non-human antibodies are well known in the art. Generally, a humanized antibody has one or more amino acid residues introduced into it from a source that is non-human. These non-human amino acid residues are often referred to as “import” residues, which are typically taken from an “import” variable domain. Humanization can be essentially performed following the method of Winter and co-workers (Jones et al., Nature, 321:522-525 (1986); Riechmann et al., Nature, 332:323-327 (1988); Verhoeyen et al., Science, 239:1534-1536 (1988)), by substituting rodent CDRs or CDR sequences for the corresponding sequences of a human antibody. Accordingly, such “humanized” antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

The choice of human variable domains, both light and heavy, to be used in making the humanized antibodies is very important in order to reduce antigenicity. According to the “best-fit” method, the sequence of the variable domain of a rodent antibody is screened against the entire library of known human variable domain sequences. The human sequence which is closest to that of the rodent is then accepted as the human framework (FR) for the humanized antibody (Sims et al., J. Immunol., 151:2296 (1993) and Chothia et al., J. Mol. Biol., 196:901 (1987)). Another method uses a particular framework derived from the consensus sequence of all human antibodies of a particular subgroup of light or heavy chains. The same framework may be used for several different humanized antibodies (Carter et al., Proc. Natl. Acad. Sci. USA, 89:4285 (1992); Presta et al., J. Immunol., 151:2623 (1993)).

It is important that antibodies be humanized with retention of high affinity for the antigen and other favorable biological properties. To achieve this goal, according to one method, humanized antibodies are prepared by a process of analysis of the parental sequences and various conceptual humanized products using three dimensional models of the parental and humanized sequences. Three dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this way, FR residues can be selected and combined from the consensus and import sequence so that the desired antibody characteristic, such as increased affinity for the target antigen(s), is achieved. In general, the CDR residues are directly and most substantially involved in influencing antigen binding (see, WO 94/04679, published 3 Mar. 1994).

Transgenic animals (e.g., mice) that are capable, upon immunization, of producing a full repertoire of human antibodies in the absence of endogenous immunoglobulin production can be employed. For example, it has been described that the homozygous deletion of the antibody heavy chain joining region (J(H)) gene in chimeric and germ-line mutant mice results in complete inhibition of endogenous antibody production. Transfer of the human germ-line immunoglobulin gene array in such germ-line mutant mice will result in the production of human antibodies upon antigen challenge (see, e.g., Jakobovits et al., Proc. Natl. Acad. Sci. USA, 90:2551-255 (1993); Jakobovits et al., Nature, 362:255-258 (1993); Bruggemann et al., Year in Immuno., 7:33 (1993)). Human antibodies can also be produced in phage display libraries (Hoogenboom et al., J. Mol. Biol., 227:381 (1991); Marks et al., J. Mol. Biol., 222:581 (1991)). The techniques of Cote et al. and Boerner et al. are also available for the preparation of human monoclonal antibodies (Cole et al., Monoclonal Antibodies and Cancer Therapy, Alan R. Liss, p. 77 (1985); Boerner et al., J. Immunol., 147(1):86-95 (1991)).

The present invention further provides a hybridoma cell that produces the monoclonal antibody of the invention. An example of such a hybridoma cell is a hybridoma cell which produces a monoclonal antibody that specifically binds an epitope contained within amino acids 1-124 of the SKCG-1 polypeptide (SEQ ID NO: 1).

The term “monoclonal antibody” as used herein refers to an antibody obtained from a substantially homogeneous population of antibodies, i.e., the individual antibodies comprising the population are identical except for possible naturally occurring mutations that may be present in minor amounts. The monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired activity (See, U.S. Pat. No. 4,816,567 and Morrison et al., Proc. Natl. Acad. Sci. USA, 81:6851-6855 (1984)).

Monoclonal antibodies of the invention may be prepared using hybridoma methods, such as those described by Kohler and Milstein, Nature, 256:495 (1975) or Harlow and Lane. Antibodies, A Laboratory Manual. Cold Spring Harbor Publications, New York, (1988). In a hybridoma method, a mouse or other appropriate host animal, is typically immunized with an immunizing agent to elicit lymphocytes that produce or are capable of producing antibodies that will specifically bind to the immunizing agent. Alternatively, the lymphocytes may be immunized in vitro. Preferably, the immunizing agent comprises a polypeptide of the present invention. Thus, the antibodies of the present invention specifically bind SKCG-1 protein.

Traditionally, the generation of monoclonal antibodies has depended on the availability of purified protein or peptides for use as the immunogen. More recently DNA based immunizations have shown promise as a way to elicit strong immune responses and generate monoclonal antibodies. In this approach, DNA-based immunization can be used, wherein DNA encoding a portion of SKCG-1 protein, for example, expressed as a fusion protein with human IgG1 is injected into the host animal according to methods known in the art (e.g., Kilpatrick K B, et al. Gene gun delivered DNA-based immunizations mediate rapid production of murine monoclonal antibodies to the Flt-3 receptor. Hybridoma. 1998 December; 17(6):569-76; Kilpatrick K E et al. High-affinity monoclonal antibodies to PED/PEA-15 generated using 5 microg of DNA. Hybridoma. 2000 August; 19(4):297-302, which are incorporated herein by reference in full for the methods of antibody production).

An alternate approach to immunizations with either purified protein or DNA is to use antigen expressed in baculovirus. The advantages to this system include ease of generation, high levels of expression, and post-translational modifications that are highly similar to those seen in mammalian systems. Use of this system involves expressing, for example, domains of a SKCG-1 protein antibody as fusion proteins. The antigen is produced by inserting a gene fragment in-frame between the signal sequence and the mature protein domain, for example, of the SKCG-1 protein antibody nucleotide sequence. This results in the display of the foreign proteins on the surface of the virion. This method allows immunization with whole virus, eliminating the need for purification of target antigens.

Generally, either peripheral blood lymphocytes (“PBLs”) are used in methods of producing monoclonal antibodies if cells of human origin are desired, or spleen cells or lymph node cells are used if non-human mammalian sources are desired. The lymphocytes are then fused with an immortalized cell line using a suitable fusing agent, such as polyethylene glycol, to form a hybridoma cell (Goding, “Monoclonal Antibodies: Principles and Practice” Academic Press, (1986) pp. 59-103). Immortalized cell lines are usually transformed mammalian cells, including myeloma cells of rodent, bovine, equine, and human origin. Usually, rat or mouse myeloma cell lines are employed. The hybridoma cells may be cultured in a suitable culture medium that preferably contains one or more substances that inhibit the growth or survival of the unfused, immortalized cells. For example, if the parental cells lack the enzyme hypoxanthine guanine phosphoribosyl transferase (HGPRT or HPRT), the culture medium for the hybridomas typically will include hypoxanthine, aminopterin, and thymidine (“HAT medium”), which substances prevent the growth of HGPRT-deficient cells. Preferred immortalized cell lines are those that fuse efficiently, support stable high level expression of antibody by the selected antibody-producing cells, and are sensitive to a medium such as HAT medium. More preferred immortalized cell lines are murine myeloma lines, which can be obtained, for instance, from the Salk Institute Cell Distribution Center, San Diego, Calif. and the American Type Culture Collection, Rockville, Md. Human myeloma and mouse-human heteromyeloma cell lines also have been described for the production of human monoclonal antibodies (Kozbor, J. Immunol., 133:3001 (1984); Brodeur et al., “Monoclonal Antibody Production Techniques and Applications” Marcel Dekker, Inc., New York, (1987) pp. 51-63). The culture medium in which the hybridoma cells are cultured can then be assayed for the presence of monoclonal antibodies directed against SKCG-1 protein. Preferably, the binding specificity of monoclonal antibodies produced by the hybridoma cells is determined by immunoprecipitation or by an in vitro binding assay, such as radioimmunoassay (RIA) or enzyme-linked immunoabsorbent assay (ELISA). Such techniques and assays are known in the art, and are described further in the Examples below or in Harlow and Lane “Antibodies, A Laboratory Manual” Cold Spring Harbor Publications, New York, (1988).

After the desired hybridoma cells are identified, the clones may be subcloned by limiting dilution or FACS sorting procedures and grown by standard methods. Suitable culture media for this purpose include, for example, Dulbecco's Modified Eagle's Medium and RPMI-1640 medium. Alternatively, the hybridoma cells may be grown in vivo as ascites in a mammal.

The monoclonal antibodies secreted by the subclones may be isolated or purified from the culture medium or ascites fluid by conventional immunoglobulin purification procedures such as, for example, protein A-Sepharose, protein G, hydroxylapatite chromatography, gel electrophoresis, dialysis, or affinity chromatography.

The monoclonal antibodies may also be made by recombinant DNA methods, such as those described in U.S. Pat. No. 4,816,567. DNA encoding the monoclonal antibodies of the invention can be readily isolated and sequenced using conventional procedures (e.g., by using oligonucleotide probes that are capable of binding specifically to genes encoding the heavy and light chains of murine antibodies). The hybridoma cells of the invention serve as a preferred source of such DNA. Once isolated, the DNA may be placed into expression vectors, which are then transfected into host cells such as simian COS cells, Chinese hamster ovary (CHO) cells, plasmacytoma cells, or myeloma cells that do not otherwise produce immunoglobulin protein, to obtain the synthesis of monoclonal antibodies in the recombinant host cells. The DNA also may be modified, for example, by substituting the coding sequence for human heavy and light chain constant domains in place of the homologous murine sequences (U.S. Pat. No. 4,816,567) or by covalently joining to the immunoglobulin coding sequence all or part of the coding sequence for a non-immunoglobulin polypeptide. Optionally, such a non-immunoglobulin polypeptide is substituted for the constant domains of an antibody of the invention or substituted for the variable domains of one antigen-combining site of an antibody of the invention to create a chimeric bivalent antibody comprising one antigen-combining site having specificity for SKCG-1 polypeptide and another antigen-combining site having specificity for a different antigen.

The present invention further provides an isolated nucleic acid, comprising a nucleotide sequence that encodes an immunoglobulin heavy chain or fragment thereof of an antibody or fragment thereof of an antibody that specifically binds an epitope contained within amino acids 1-124 of the SKCG-1 protein (SEQ ID NO: 1). Vectors comprising any of the nucleic acids comprising a nucleotide sequence that encodes an immunoglobulin heavy chain or a fragment thereof are also contemplated by this invention as are host cells comprising such vectors. Suitable vectors and host cells for expression of the immunoglobulin heavy chains of the present invention are described above.

The present invention further provides an isolated nucleic acid, comprising a nucleotide sequence that encodes an immunoglobulin light chain or fragment thereof of an antibody or fragment thereof of an antibody that specifically binds an epitope contained within amino acids 1-124 of the SKCG-1 protein (SEQ ID NO: 1). Vectors comprising any of the nucleic acids comprising a nucleotide sequence that encodes an immunoglobulin light chain or a fragment thereof are also contemplated by this invention as are host cells comprising such vectors. Suitable vectors and host cells for expression of the immunoglobulin light chains of the present invention are described above.

Further provided by this invention is a purified polypeptide, comprising an amino acid sequence of an immunoglobulin heavy chain or a fragment thereof of an antibody that binds an epitope contained within amino acids 1-124 of the SKCG-1 protein (SEQ ID NO: 1). Methods of obtaining polypeptides comprising an amino acid sequence of an immunoglobulin heavy chain and polypeptides comprising an amino acid sequence of an immunoglobulin heavy chain are described above.

Further provided by this invention is a purified polypeptide, comprising an amino acid sequence of an immunoglobulin light chain or a fragment thereof of an antibody that binds an epitope contained within amino acids 1-124 of the SKCG-1 protein (SEQ ID NO: 1). Methods of obtaining polypeptides comprising an amino acid sequence of an immunoglobulin light chain and polypeptides comprising an amino acid sequence of an immunoglobulin light chain are described above.

In vitro methods are also suitable for preparing monovalent antibodies. Digestion of antibodies to produce fragments thereof, particularly, Fab fragments, can be accomplished using routine techniques known in the art. For instance, digestion can be performed using papain. Examples of papain digestion are described in WO 94/29348 published Dec. 22, 1994, U.S. Pat. No. 4,342,566, and Harlow and Lane, Antibodies, A Laboratory Manual, Cold Spring Harbor Publications, New York, (1988). Papain digestion of antibodies typically produces two identical antigen binding fragments, called Fab fragments, each with a single antigen binding site, and a residual Fc fragment. Pepsin treatment yields a fragment, called the F(ab′)2 fragment, that has two antigen combining sites and is still capable of cross-linking antigen.

The Fab fragments produced in the antibody digestion also contain the constant domains of the light chain and the first constant domain of the heavy chain. Fab′ fragments differ from Fab fragments by the addition of a few residues at the carboxy terminus of the heavy chain domain including one or more cysteines from the antibody hinge region. The F(ab′)2 fragment is a bivalent fragment comprising two Fab′ fragments linked by a disulfide bridge at the hinge region. Fab′-SH is the designation herein for Fab′ in which the cysteine residue(s) of the constant domains bear a free thiol group. Antibody fragments originally were produced as pairs of Fab′ fragments which have hinge cysteines between them. Other chemical couplings of antibody fragments are also known.

An isolated immunogenically specific paratope or fragment of the antibody is also provided. A specific immunogenic epitope of the antibody can be isolated from the whole antibody by chemical or mechanical disruption of the molecule. The purified fragments thus obtained are tested to determine their immunogenicity and specificity by the methods taught herein. Immunoreactive paratopes of the antibody, optionally, are synthesized directly. An immunoreactive fragment is defined as an amino acid sequence of at least about two to five consecutive amino acids derived from the antibody amino acid sequence.

One method of producing proteins comprising the antibodies of the present invention is to link two or more peptides or polypeptides together by protein chemistry techniques. For example, peptides or polypeptides can be chemically synthesized using currently available laboratory equipment using either Fmoc (9-fluorenylmethyloxycarbonyl) or Boc (tert-butyloxycarbonoyl) chemistry. (Applied Biosystems, Inc., Foster City, Calif.). One skilled in the art can readily appreciate that a peptide or polypeptide corresponding to the antibody of the present invention, for example, can be synthesized by standard chemical reactions. For example, a peptide or polypeptide can be synthesized and not cleaved from its synthesis resin, whereas the other fragment of an antibody can be synthesized and subsequently cleaved from the resin, thereby exposing a terminal group which is functionally blocked on the other fragment. By peptide condensation reactions, these two fragments can be covalently joined via a peptide bond at their carboxyl and amino termini, respectively, to form an antibody, or fragment thereof. (Grant GA (1992) Synthetic Peptides: A User Guide. W. H. Freeman and Co., N.Y. (1992); Bodansky M and Trost B., Ed. (1993) Principles of Peptide Synthesis. Springer-Verlag Inc., NY. Alternatively, the peptide or polypeptide is independently synthesized in vivo as described above. Once isolated, these independent peptides or polypeptides may be linked to form an antibody or fragment thereof via similar peptide condensation reactions.

For example, enzymatic ligation of cloned or synthetic peptide segments allow relatively short peptide fragments to be joined to produce larger peptide fragments, polypeptides or whole protein domains (Abrahmsen L et al., Biochemistry, 30:4151 (1991)). Alternatively, native chemical ligation of synthetic peptides can be utilized to synthetically construct large peptides or polypeptides from shorter peptide fragments. This method consists of a two-step chemical reaction (Dawson et al. Synthesis of Proteins by Native Chemical Ligation. Science, 266:776-779 (1994)). The first step is the chemoselective reaction of an unprotected synthetic peptide-alpha-thioester with another unprotected peptide segment containing an amino-terminal Cys residue to give a thioester-linked intermediate as the initial covalent product. Without a change in the reaction conditions, this intermediate undergoes spontaneous, rapid intramolecular reaction to form a native peptide bond at the ligation site. Application of this native chemical ligation method to the total synthesis of a protein molecule is illustrated by the preparation of human interleukin 8 (IL-8) (Baggiolini M et al. (1992) FEBS Lett. 307:97-101; Clark-Lewis I et al., J. Biol. Chem., 269:16075 (1994); Clark-Lewis I et al., Biochemistry, 30:3128 (1991); Rajarathnam K et al., Biochemistry 33:6623-30 (1994)).

Alternatively, unprotected peptide segments are chemically linked where the bond formed between the peptide segments as a result of the chemical ligation is an unnatural (non-peptide) bond (Schnolzer, M et al. Science, 256:221 (1992)). This technique has been used to synthesize analogs of protein domains as well as large amounts of relatively pure proteins with full biological activity (deLisle Milton R C et al., Techniques in Protein Chemistry IV. Academic Press, New York, pp. 257-267 (1992)).

The invention also provides fragments of antibodies which have bioactivity. The polypeptide fragments of the present invention can be recombinant proteins obtained by cloning nucleic acids encoding the polypeptide in an expression system capable of producing the polypeptide fragments thereof, such as an adenovirus or baculovirus expression system. For example, one can determine the active domain of an antibody from a specific hybridoma that can cause a biological effect associated with the interaction of the antibody with SKCG-1 protein. For example, amino acids found not to contribute to either the activity or the binding specificity or affinity of the antibody can be deleted without a loss in the respective activity. For example, in various embodiments, amino or carboxy-terminal amino acids are sequentially removed from either the native or the modified non-immunoglobulin molecule or the immunoglobulin molecule and the respective activity assayed in one of many available assays. In another example, a fragment of an antibody comprises a modified antibody wherein at least one amino acid has been substituted for the naturally occurring amino acid at a specific position, and a portion of either amino terminal or carboxy terminal amino acids, or even an internal region of the antibody, has been replaced with a polypeptide fragment or other moiety, such as biotin, which can facilitate in the purification of the modified antibody. For example, a modified antibody can be fused to a maltose binding protein, through either peptide chemistry or cloning the respective nucleic acids encoding the two polypeptide fragments into an expression vector such that the expression of the coding region results in a hybrid polypeptide. The hybrid polypeptide can be affinity purified by passing it over an amylose affinity column, and the modified antibody receptor can then be separated from the maltose binding region by cleaving the hybrid polypeptide with the specific protease factor Xa. (See, for example, New England Biolabs Product Catalog, 1996, pg. 164.). Similar purification procedures are available for isolating hybrid proteins from eukaryotic cells as well.

The fragments, whether attached to other sequences or not, include insertions, deletions, substitutions, or other selected modifications of particular regions or specific amino acids residues, provided the activity of the fragment is not significantly altered or impaired compared to the nonmodified antibody or antibody fragment. These modifications can provide for some additional property, such as to remove or add amino acids capable of disulfide bonding, to increase its bio-longevity, to alter its secretory characteristics, etc. In any case, the fragment must possess a bioactive property, such as binding activity, regulation of binding at the binding domain, etc. Functional or active regions of the antibody may be identified by mutagenesis of a specific region of the protein, followed by expression and testing of the expressed polypeptide. Such methods are readily apparent to a skilled practitioner in the art and can include site-specific mutagenesis of the nucleic acid encoding the antigen. (Zoller M J et al. Nucl. Acids Res. 10:6487-500 (1982).

A variety of immunoassay formats may be used to select antibodies that selectively bind with a particular protein, variant, or fragment. For example, solid-phase ELISA immunoassays are routinely used to select antibodies selectively immunoreactive with a protein, protein variant, or fragment thereof. See Harlow and Lane. Antibodies, A Laboratory Manual. Cold Spring Harbor Publications, New York, (1988), for a description of immunoassay formats and conditions that could be used to determine selective binding. The binding affinity of a monoclonal antibody can, for example, be determined by the Scatchard analysis of Munson et al., Anal. Biochem., 107:220 (1980).

Also provided is an antibody reagent kit comprising containers of a monoclonal antibody or fragment thereof of the invention and one or more reagents for detecting binding of the antibody or fragment thereof to a SKCG-1 polypeptide. The reagents can include, for example, fluorescent tags, enzymatic tags, or other tags. The reagents can also include secondary or tertiary antibodies or reagents for enzymatic reactions, wherein the enzymatic reactions produce a product that can be visualized.

Detection Methods

The present invention provides a method for detecting expression of a tumor suppressor gene in a subject, comprising analyzing a sample from the subject for expression of a SKCG-1 gene. As utilized herein, “expression” refers to the transcription of a SKCG-1 gene to yield a SKCG-1 nucleic acid, such as SKCG-1 mRNA. The term “expression” also refers to the transcription and translation of a gene to yield the encoded protein, in particular a SKCG-1 polypeptide or a fragment thereof. Therefore, one of skill in the art can detect the expression of SKCG-1 by monitoring SKCG-1 nucleic acid production and/or expression of the SKCG-1 protein.

The amount of nucleic acid encoding SKCG-1 in a cell can be determined by methods standard in the art for detecting or quantitating nucleic acid in a cell, such as in situ hybridization, quantitative PCR, Northern blotting, ELISPOT, dot blotting, etc., as well as any other method now known or later developed for detecting or quantitating the amount of a nucleic acid in a cell.

The presence or amount of SKCG-1 protein in a cell can be determined by methods standard in the art, such as Western blotting, ELISA, ELISPOT, immunoprecipitation, immunofluorescence (e.g., FACS), immunohistochemistry, immunocytochemistry, etc., as well as any other method now known or later developed for detecting or quantitating protein in or produced by a cell.

The sample of this invention can be from any organism and can be, but is not limited to, peripheral blood, bone marrow specimens, primary tumors, embedded tissue sections, frozen tissue sections, cell preparations, cytological preparations, exfoliate samples (e.g., sputum), fine needle aspirations, lung fluid, amnion cells, fresh tissue, dry tissue, and cultured cells or tissue. The sample can be from malignant tissue or non-malignant tissue. The sample can be unfixed or fixed according to standard protocols widely available in the art and can also be embedded in a suitable medium for preparation of the sample. For example, the sample can be embedded in paraffin or other suitable medium (e.g., epoxy or acrylamide) to facilitate preparation of the biological specimen for the detection methods of this invention. Furthermore, the sample can be embedded in any commercially available mounting medium, either aqueous or organic.

The sample can be on, supported by, or attached to, a substrate which facilitates detection. A substrate of the present invention can be, but is not limited to, a microscope slide, a culture dish, a culture flask, a culture plate, a culture chamber, ELISA plates, as well as any other substrate that can be used for containing or supporting biological samples for analysis according to the methods of the present invention. The substrate can be of any material suitable for the purposes of this invention, such as, for example, glass, plastic, polystyrene, mica and the like. The substrates of the present invention can be obtained from commercial sources or prepared according to standard procedures well known in the art.

Conversely, an antibody or fragment thereof, an antigenic fragment of SKCG-1, or SKCG-1 nucleic acid of the invention can be on, supported by, or attached to a substrate which facilitates detection. Such a substrate can include a chip, a microarray or a mobile solid support. Thus, provided by the invention are substrates including one or more of the antibodies or antibody fragments, antigenic fragments of SKCG-1 polypeptides, or SKCG-1 nucleic acids of the invention.

The nucleic acids of this invention can be detected with a probe capable of hybridizing to the nucleic acid of a cell or a sample. This probe can be a nucleic acid comprising the nucleotide sequence of a coding strand or its complementary strand or the nucleotide sequence of a sense strand or antisense strand, or a fragment thereof. The nucleic acid can comprise the nucleic acid of the SKCG-1 gene or fragments thereof. Thus, the probe of this invention can be either DNA or RNA and can bind either DNA or RNA, or both, in the biological sample. The probe can be the coding or complementary strand of a complete SKCG-1 gene or SKCG-1 gene fragment.

The nucleic acids of the present invention, for example, SEQ ID NO: 2, SEQ ID NO: 3 and fragments thereof, can be utilized as probes or primers to detect SKCG-1 nucleic acids. For example, a polynucleotide probe or primer comprising a polynucleotide selected from the group consisting of at least 25 contiguous nucleotides of SEQ ID NO: 2 and at least 25 contiguous nucleotides of SEQ ID NO: 3 can be utilized to detect a SKCG-1 nucleic acid. Therefore, the polynucleotide probes or primers of this invention can be at least 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195 or at least 200 nucleotides in length.

As used herein, the term “nucleic acid probe” refers to a nucleic acid fragment that selectively hybridizes under stringent conditions with a nucleic acid comprising a nucleic acid set forth in a sequence listed herein. This hybridization must be specific. The degree of complementarity between the hybridizing nucleic acid and the sequence to which it hybridizes should be at least enough to exclude hybridization with a nucleic acid encoding an unrelated protein.

Stringent conditions refers to the washing conditions used in a hybridization protocol. In general, the washing conditions should be a combination of temperature and salt concentration chosen so that the denaturation temperature is approximately 5-20° C. below the calculated Tₘ of the nucleic acid hybrid under study. The temperature and salt conditions are readily determined empirically in preliminary experiments in which samples of reference DNA immobilized on filters are hybridized to the probe or protein coding nucleic acid of interest and then washed under conditions of different stringencies. The Tₘ of such an oligonucleotide can be estimated by allowing 2° C. for each A or T nucleotide, and 4° C. for each G or C. For example, an 18 nucleotide probe of 50% G+C would, therefore, have an approximate Tₘ of 54° C.

Stringent conditions are known to one of skill in the art. See, for example, Sambrook et al. (2001).

The following is an exemplary set of hybridization conditions and is not limiting:
Very High Stringency
Hybridization: 5×SSC at 65° C. for 16 hours
Wash twice: 2×SSC at room temperature (RT) for 15 minutes each
Wash twice: 0.5×SSC at 65° C. for 20 minutes each
High Stringency
Hybridization: 5×-6×SSC at 65° C.-70° C. for 16-20 hours
Wash twice: 2×SSC at RT for 5-20 minutes each
Wash twice: 1×SSC at 55° C.-70° C. for 30 minutes each
Low Stringency
Hybridization: 6×SSC at RT to 55° C. for 16-20 hours
Wash at least twice: 2×-3×SSC at RT to 55° C. for 20-30 minutes each.

As mentioned above, the SKCG-1 nucleic acids and fragments thereof can be utilized as primers to amplify a SKCG-1 nucleic acid, such as a SKCG-1 gene transcript, by standard amplification techniques. For example, expression of a SKCG-1 gene transcript can be quantified by RT-PCR using RNA isolated from cells, as described in the Examples.

A variety of PCR techniques are familiar to those skilled in the art. For a review of PCR technology, see White (1997) and the publication entitled “PCR Methods and Applications” (1991, Cold Spring Harbor Laboratory Press), which is incorporated herein by reference in its entirety for amplification methods. In each of these PCR procedures, PCR primers on either side of the nucleic acid sequences to be amplified are added to a suitably prepared nucleic acid sample along with dNTPs and a thermostable polymerase such as Taq polymerase, Pfu polymerase, or Vent polymerase. The nucleic acid in the sample is denatured and the PCR primers are specifically hybridized to complementary nucleic acid sequences in the sample. The hybridized primers are extended. Thereafter, another cycle of denaturation, hybridization, and extension is initiated. The cycles are repeated multiple times to produce an amplified fragment containing the nucleic acid sequence between the primer sites. PCR has further been described in several patents including U.S. Pat. Nos. 4,683,195, 4,683,202 and 4,965,188. Each of these publications is incorporated herein by reference in its entirety for PCR methods. One of skill in the art would know how to design and synthesize primers that amplify SEQ ID NO: 2, SEQ ID NO: 3 or a fragment thereof.

A detectable label may be included in an amplification reaction. Suitable labels include fluorochromes, e.g. fluorescein isothiocyanate (FITC), rhodamine, Texas Red, phycoerythrin, allophycocyanin, 6-carboxyfluorescein (6-FAM), 2′,7′-dimethoxy-4′,5′-dichloro-6-carboxyfluorescein (JOE), 6-carboxy-X-rhodamine (ROX), 6-carboxy-2′,4′,7′,4,7-hexachlorofluorescein (HEX), 5-carboxyfluorescein (5-FAM) or N,N,N′,N′-tetramethyl-6-carboxyrhodamine (TAMRA), radioactive labels, e.g., ³²P, ³⁵S, ³H; etc. The label may be a two stage system, where the amplified DNA is conjugated to biotin, haptens, etc. having a high affinity binding partner, e.g. avidin, specific antibodies, etc., where the binding partner is conjugated to a detectable label. The label may be conjugated to one or both of the primers. Alternatively, the pool of nucleotides used in the amplification is labeled, so as to incorporate the label into the amplification product.

The sample nucleic acid, e.g. amplified fragment, can be analyzed by one of a number of methods known in the art. The nucleic acid can be sequenced by dideoxy or other methods. Hybridization with the sequence can also be used to determine its presence, by Southern blots, dot blots, etc.

The SKCG-1 nucleic acids of the invention can also be used in polynucleotide arrays. Polynucleotide arrays provide a high throughput technique that can assay a large number of polynucleotide sequences in a single sample. This technology can be used, for example, as a diagnostic tool to identify samples with reduced expression of SKCG-1 as compared to a control sample.

To create arrays, single-stranded polynucleotide probes can be spotted onto a substrate in a two-dimensional matrix or array. Each single-stranded polynucleotide probe can comprise at least 6, 7, 8, 9, 10, 11, 12, 13, 14, 15, 16, 17, 18, 19, 20, 25, or 30 or more contiguous nucleotides selected from the nucleotide sequences shown in SEQ ID NOS: 2 and 3.

The substrate can be any substrate to which polynucleotide probes can be attached, including but not limited to glass, nitrocellulose, silicon, and nylon. Polynucleotide probes can be bound to the substrate by either covalent bonds or by non-specific interactions, such as hydrophobic interactions. Techniques for constructing arrays and methods of using these arrays are described in EP No. 0 799 897; PCT No. WO 97/29212; PCT No. WO 97/27317; EP No. 0 785 280; PCT No. WO 97/02357; U.S. Pat. Nos. 5,593,839; 5,578,832; EP No. 0 728 520; U.S. Pat. No. 5,599,695; EP No. 0 721 016; U.S. Pat. No. 5,556,752; PCT No. WO 95/22058; and U.S. Pat. No. 5,631,734. Commercially available polynucleotide arrays, such as Affymetrix GeneChip™, can also be used. Use of the GeneChip™ to detect gene expression is described, for example, in Lockhart et al., Nature Biotechnology 14:1675 (1996); Chee et al., Science 274:610 (1996); Hacia et al., Nature Genetics 14:441, 1996; and Kozal et al., Nature Medicine 2:753, 1996.

Tissue samples can be treated to form single-stranded polynucleotides, for example by heating or by chemical denaturation, as is known in the art. The single-stranded polynucleotides in the tissue sample can then be labeled and hybridized to the polynucleotide probes on the array. Detectable labels which can be used include but are not limited to radiolabels, biotinylated labels, fluorophors, and chemiluminescent labels. Double stranded polynucleotides, comprising the labeled sample polynucleotides bound to polynucleotide probes, can be detected once the unbound portion of the sample is washed away. Detection can be visual or with computer assistance.

The present invention also provides methods of detecting a SKCG-1 polypeptide or fragment thereof. The antibody utilized to detect a SKCG-1 polypeptide, or fragment thereof, can be linked to a detectable label either directly or indirectly through use of a secondary and/or tertiary antibody; thus, bound antibody, fragment or molecular complex can be detected directly in an ELISA or similar assay.

The sample can be on, supported by, or attached to, a substrate which facilitates detection. A substrate of the present invention can be, but is not limited to, a microscope slide, a culture dish, a culture flask, a culture plate, a culture chamber, ELISA plates, as well as any other substrate that can be used for containing or supporting biological samples for analysis according to the methods of the present invention. The substrate can be of any material suitable for the purposes of this invention, such as, for example, glass, plastic, polystyrene, mica and the like. The substrates of the present invention can be obtained from commercial sources or prepared according to standard procedures well known in the art.

Conversely, an antibody or fragment thereof, an antigenic fragment of a SKCG-1 polypeptide can be on, supported by, or attached to a substrate which facilitates detection. Such a substrate can be a mobile solid support. Thus, provided by the invention are substrates including one or more of the antibodies or antibody fragments, or antigenic fragments of a SKCG-1 polypeptide.

In addition to utilizing the detection methods of the present invention to detect the presence of and quantify the amount of SKCG-1 nucleic acid and/or SKCG-1 protein present in a sample, the detection methods of the present invention can be utilized to detect reduced expression of SKCG-1, wherein reduced expression of the SKCG-1 gene indicates the subject has or is at risk for developing cancer. Therefore, the present invention provides a method for determining whether a subject has or is at risk for developing cancer comprising: detecting reduced expression of the SKCG-1 gene in a test sample from the subject as compared to a control sample, wherein reduced expression of the SKCG-1 gene in the test sample indicates that the subject has or is at risk for developing cancer. The reduction in expression of the SKCG-1 gene does not have to be complete and can range from a decrease in the expression of the SKCG-1 gene to elimination of SKCG-1 gene expression.

The term cancer, when used herein refers to or describes the physiological condition, preferably in a mamalian subject, that is typically characterized by unregulated cell growth. Examples include but are not limited to Acute Childhood Lymphoblastic Leukemia, Acute Lymphoblastic Leukemia, Acute Lymphocytic Leukemia, Acute Myeloid Leukemia, Adrenal carcinoma, Adrenocortical Carcinoma, Adult (Primary) Hepatocellular Cancer, Adult (Primary) Liver Cancer, Adult Acute Lymphocytic Leukemia, Adult Acute Myeloid Leukemia, Adult Hodgkin's Disease, Adult Hodgkin's Lymphoma, Adult Lymphocytic Leukemia, Adult Non-Hodgkin's Lymphoma, Adult Primary Liver Cancer, Adult Soft Tissue Sarcoma, AIDS-Related Lymphoma, AIDS-Related Malignancies, Anal Cancer, Astrocytoma, Bile Duct Cancer, Bladder Cancer, Bone Cancer, Brain Stem Glioma, Brain Tumors, Breast Cancer, Cancer of the Renal Pelvis and Ureter, Central Nervous System (Primary) Lymphoma, Central Nervous System Lymphoma, Cerebellar Astrocytoma, Cerebral Astrocytoma, Cervical Cancer, Childhood (Primary) Hepatocellular Cancer, Childhood (Primary) Liver Cancer, Childhood Acute Lymphoblastic Leukemia, Childhood Acute Myeloid Leukemia, Childhood Brain Stem Glioma, Childhood Cerebellar Astrocytoma, Childhood Cerebral Astrocytoma, Childhood Extracranial Germ Cell Tumors, Childhood Hodgkin's Disease, Childhood Hodgkin's Lymphoma, Childhood Hypothalamic and Visual Pathway Glioma, Childhood Lymphoblastic Leukemia, Childhood Medulloblastoma, Childhood Non-Hodgkin's Lymphoma, Childhood Pineal and Supratentorial Primitive Neuroectodermal Tumors, Childhood Primary Liver Cancer, Childhood Rhabdomyosatcoma, Childhood Soft Tissue Sarcoma, Childhood Visual Pathway and Hypothalamic Glioma, Chronic Lymphocytic Leukemia, Chronic Myelogenous Leukemia, Colon Cancer, Cutaneous T-Cell Lymphoma, Endocrine Pancreas Islet Cell Carcinoma, Endometrial Cancer, Ependymoma, Epithelial Cancer, Esophageal Cancer, Estrogen induced breast cancer, Ewing's Sarcoma and Related Tumors, Exocrine Pancreatic Cancer, Extracranial Germ Cell Tumor, Extragonadal Germ Cell Tumor, Extrahepatic Bile Duct Cancer, Eye Cancer, Female Breast Cancer, Gaucher's Disease, Gallbladder Cancer, Gastric Cancer, Gastrointestinal Carcinoid Tumor, Gastrointestinal Tumors, Germ Cell Tumors, Gestational Trophoblastic Tumor, Hairy Cell Leukemia, Head and Neck Cancer, Hepatocellular Cancer, Hodgkin's Disease, Hodgkin's Lymphoma, Hypergammaglobulinemia, Hypopharyngeal Cancer, Intestinal Cancers, Intraocular Melanoma, Islet Cell Carcinoma, Islet Cell Pancreatic Cancer, Kaposi's Sarcoma, Kidney Cancer, Laryngeal Cancer, Lip and Oral Cavity Cancer, Liver Cancer, Lung Cancer, Lymphoproliferative Disorders, Lymphomas, Macroglobulinemia, Male Breast Cancer, Malignant Mesothelioma, Malignant Thymoma, Medulloblastoma, Melanoma, Mesothelioma, Metastatic Occult Primary Squamous Neck Cancer, Metastatic Primary Squamous Neck Cancer, Metastatic Squamous Neck Cancer, Multiple Myeloma, Multiple Myeloma/Plasma Cell Neoplasm, Myelodysplastic Syndrome, Myelogenous Leukemia, Myeloid Leukemia, Myeloproliferative Disorders, Nasal Cavity and Paranasal Sinus Cancer, Nasopharyngeal Cancer, Neuroblastoma, Non-Hodgkin's Lymphoma During Pregnancy, Nonmelanoma Skin Cancer, Non-Small Cell Lung Cancer, Occult Primary Metastatic Squamous Neck Cancer, Oropharyngeal Cancer, Osteo-/Malignant Fibrous Sarcoma, Osteosarcoma/Malignant Fibrous Histiocytoma, Osteosarcoma/Malignant Fibrous Histiocytoma of Bone, Ovarian Cancers (such as Ovarian Epithelial Cancer, Ovarian Germ Cell Tumor, Ovarian Low Malignant Potential Tumor), Pancreatic Cancer, Paraproteinemias, Purpura, Parathyroid Cancer, Penile Cancer, Pheochromocytoma, Pituitary Tumor, Plasma Cell Neoplasm/Multiple Myeloma, Primary Central Nervous System Lymphoma, Primary Liver Cancer, Prostate Cancer, Ras induced cancer, Rectal Cancer, Renal Cell Cancer, Renal Pelvis and Ureter Cancer, Retinoblastoma, Rhabdomyosarcoma, Salivary Gland Cancer, Sarcoidosis Sarcomas, Sezary Syndrome, Skin Cancer, Small Cell Lung Cancer, Small Intestine Cancer, Soft Tissue Sarcoma, Squamous Neck Cancer, Squamous cell carcinoma, Stomach Cancer, Supratentorial Primitive Neuroectodermal and Pineal Tumors, T-Cell Lymphoma, Testicular Cancer, Thymoma, Thyroid Cancer, Transitional Cell Cancer of the Renal Pelvis and Ureter, Transitional Renal Pelvis and Ureter Cancer, Trophoblastic Tumors, Ureter and Renal Pelvis Cell Cancer, Urethral Cancer, Uterine Cancer, Uterine Sarcoma, Vaginal Cancer, Visual Pathway and Hypothalamic Glioma, Vulvar Cancer, Waldenstrom's Macroglobulinemia, Wilms' Tumor, and any other hyperproliferative disease, located in an organ listed above. More particular examples of such cancers include squamous cell carcinoma, lung cancer, pancreatic cancer, cervical cancer, bladder cancer, brain cancer, hepatoma, breast cancer, renal carcinoma, liver cancer, estrogen dependent breast cancer, urinary cancer, ovarian cancer, prostrate carcinoma, rhabdomyosarcoma, colon carcinoma, testicular cancer, adrenal cancer, and head and neck cancer. Therefore, the methods of the present invention can be utilized to detect all of the cancers described above and any other condition associated with a hyperproliferative disorder or a disorder characterized by unregulated cell growth.

Since therapy and clinical decisions are often dependent on diagnosis, detection of SKCG-1 expression utilizing the methods described herein, allows one of skill in the art to measure SKCG-1 expression levels and determine whether these levels indicate the presence of cancer or increased risk of developing cancer. One skilled in the art would be able to measure SKCG-1 levels in numerous subjects in order to establish ranges of SKCG-1 levels that correspond to clinically defined stages such as, for example, 1) normal, 2) at risk of developing cancer, 3) pre-cancerous or 4) cancerous. These stages are not intended to be limiting as one of skill in the art may define other stages depending on the type of sample, type of cancer, age of the subject and other factors.

Any of the detection methods described herein can be used in combination with other detection methods to confirm the presence or absence of cancer. For example, if SKCG-1 expression levels indicate the presence of cancer in the brain, other tests, such as a CT scan or biopsy can be performed to confirm the presence of cancer. Similary, if SKCG-1 expression levels indicate the presence of cancer in the breast, a mammogram, biopsy or other procedure can be performed to confirm the presence of cancer.

Upon detection of decreased SKCG-1 expression or correlation of SKCG-1 expression levels with cancer, the skilled practitioner can administer a therapy suited for the treatment of cancer. The subjects of this invention undergoing anti-cancer therapy can include subjects undergoing surgery, chemotherapy, radiotherapy, immunotherapy or any combination thereof. Examples of chemotherapeutic agents include cisplatin, 5-fluorouracil and S-1. Immunotherapeutics methods include administration of interleukin-2 and interferon-α.

The present invention also provides a method for determining whether a subject has or is at risk for developing cancer comprising: analyzing the methylation status of a SKCG-1 gene in a sample from the subject and detecting a difference in methylation status as compared to that of a comparable normal cell, wherein the difference in methylation status indicates that the subject has or is at risk of developing cancer.

As used herein, “a cell of a normal subject” or a “normal cell” means a cell or tissue which is histologically normal and was obtained from a subject believed to be without malignancy and having no increased risk of developing a malignancy or was obtained from tissues adjacent to tissue known to be malignant and which is determined to be histologically normal (non-malignant) as determined by a pathologist.

One of skill in the art can obtain and analyze the methylation pattern or status of a SKCG-1 gene in normal cells and in cancerous cells to create a library of SCKG-1 methylation patterns. The methylation pattern of a SKCG-1 gene in a sample from a subject can then be compared with these patterns. If the methylation pattern obtained from a sample is more similar to the methylation pattern from a cancerous cell than it is to the methylation pattern obtained from a normal cell, this is indicative of a subject that has or is at risk of developing cancer.

Also provided by the present invention is a method of diagnosing cancer comprising: a) determining methylation of a SKCG-1 gene in a sample to obtain a methylation pattern; b) comparing the methylation pattern of step a) with a known methylation pattern for a type of cancer; and c) diagnosing the type of cancer based on matching of the methylation pattern of a) with a known methylation pattern for a type of cancer.

Methods of measuring methylation are known in the art and include, but are not limited to methylation-specific PCR, methylation microarray analysis and ChIP (a chromatin immunoprecipitation approach) analysis. Methylation can also be monitored by digestion of nucleic acid sequences with methylation sensitive and non-sensitive restriction enzymes followed by Southern blotting or PCR analysis of the restriction products (See Takai et al. “Hypomethylation of LINE1 retrotransposon in human hepatocellular carcinomas, but not in surrounding liver cirrhosis” Jpn J. Clin. Oncol. 30(7) 306-309). One of skill in the art could also utilize methods in which genomic DNA is digested followed by PCR. (See, for example, Cartwright et al., “Analysis of Drosophila chromatin structure in vivo” Methods in Enzymology, Vol. 304).

Also provided by the present invention is a method for determining whether a subject has or is at risk for developing cancer comprising: detecting the presence or absence in the SKCG-1 gene of said subject a genetic polymorphism comprising the nucleotide sequence set forth in SEQ ID NO:2, with one or more point mutations, wherein the point mutations comprise one or more mutations located between and including nucleotide position 1546 to nucleotide position 1555 in the full-length cDNA sequence of SKCG-1 (SEQ ID NO: 3) and wherein the presence of the genetic polymorphism indicates that the subject has or is at risk of developing cancer.

A number of methods are available for analyzing nucleic acids for the presence of a specific sequence. For all of the methods described herein, genomic DNA can be extracted from a sample and this sample can be from any organism and can be, but is not limited to, peripheral blood, bone marrow specimens, primary tumors, embedded tissue sections, frozen tissue sections, cell preparations, cytological preparations, exfoliate samples (e.g., sputum), fine needle aspirations, amnion cells, fresh tissue, dry tissue, and cultured cells or tissue. Such samples can be obtained directly from a subject, commercially obtained or obtained via other means. Thus, the invention described herein can be utilized to analyze a nucleic acid sample that comprises genomic DNA, amplified DNA (such as a PCR product) cDNA, cRNA, a restriction fragment or any other desired nucleic acid sample. When one performs one of the herein described methods on genomic DNA, typically the genomic DNA will be treated in a manner to reduce viscosity of the DNA and allow better contact of a primer or probe with the target region of the genomic DNA. Such reduction in viscosity can be achieved by any desired methods, which are known to the skilled artisan, such as DNase treatment or shearing of the genomic DNA, preferably lightly.

If sufficient DNA is available, genomic DNA can be used directly. Alternatively, the region of interest is cloned into a suitable vector and grown in sufficient quantity for analysis. The nucleic acid may be amplified by conventional techniques, such as the polymerase chain reaction (PCR), to provide sufficient amounts for analysis. A variety of PCR techniques are familiar to those skilled in the art. For a review of PCR technology, see White (1997) and the publication entitled “PCR Methods and Applications” (1991, Cold Spring Harbor Laboratory Press), which is incorporated herein by reference in its entirety for amplification methods. In each of these PCR procedures, PCR primers on either side of the nucleic acid sequences to be amplified are added to a suitably prepared nucleic acid sample along with dNTPs and a thermostable polymerase such as Taq polymerase, Pfu polymerase, or Vent polymerase. The nucleic acid in the sample is denatured and the PCR primers are specifically hybridized to complementary nucleic acid sequences in the sample. The hybridized primers are extended. Thereafter, another cycle of denaturation, hybridization, and extension is initiated. The cycles are repeated multiple times to produce an amplified fragment containing the nucleic acid sequence between the primer sites. PCR has further been described in several patents including U.S. Pat. Nos. 4,683,195, 4,683,202 and 4,965,188. Each of these publications is incorporated herein by reference in its entirety for PCR methods. One of skill in the art would know how to design and synthesize primers flanking any of the polymorphic sites of this invention. One of skill in the art would know how to design primers accordingly to amplify any region of the SKCG-1 gene, including its promoter sequence, for the purposes of identifying a polymorphism at any nucleotide position throughout the SKCG-1 gene. Amplification may also be used to determine whether a polymorphism is present by using a primer that is specific for the polymorphism.

The present invention also provides an array of oligonucleotides for identification of polymorphisms, where discrete positions on the array are complementary to one or more of the SCKG-1 polymorphic sequences, e.g. oligonucleotides of at least 12 nt, frequently 20 nt, or larger, and including the sequence flanking the polymorphic position. Such an array may comprise a series of oligonucleotides, each of which can specifically hybridize to a different polymorphism of the present invention. Usually such an array will include at least 2 different polymorphic sequences, i.e. polymorphisms located at unique positions within the locus, and can include numerous SKCG-1 polymorphisms. Each oligonucleotide sequence on the array will usually be at least about 12 nt in length (i.e., 10-15 nt), may be the length of the provided polymorphic sequences, or may extend into the flanking regions to generate fragments of 100 to 200 nt in length.

The present invention also provides the use of the nucleic acid sequences of the invention in methods using a mobile solid support to analyze polymorphisms. See for example, WO 01/48244 which is incorporated herein by reference in its entirety for the methods taught therein.

Since applicants have provided chromosome 11q23.2 as the genomic locus for SKCG-1, one of skill in the art can analyze genomic DNA either directly or after amplification for the presence or absence of an SKCG-1 gene by sequencing this region of chromosome 11q23.2 and identifying the presence or absence of an SKCG-1 gene at this genomic location.

One of skill in the art can also utilize FISH with probes localized to chromosome 11 or probes localized to region 11q23.2 to identify the presence or absence of SKCG-1 (See Hemel and Eussen “Interchromosomal insertions. Identification of five cases and a review” Hum. Genet. 107(5):415-32 (2000); Engelen et al. “Duplication within chromosome region 15q11-q13 in a patient with similarities to Prader-Willi syndrome confirmed by region-specific and band specific fish” Genet. Couns. 10(2): 123-32 (1999); Lorda-Sanchez et al. “Proximal partial 5p trisomy resulting from a maternal (19;5) insertion” Am. J. Med. Genet. 68(4):476-80 (1997) All of these references are incorporated herein by this reference as they pertain to FISH analysis and techniques utilized FISH to identify chromosomal aberrations, such as insertions, deletions and duplications).

Methods of Reducing Cell Growth

The present invention provides methods of inhibiting cell growth by introducing a polynucleotide encoding a polypeptide having the amino acid sequence shown in SEQ ID NO: 1 or a fragment thereof into a cell, wherein expression of the polypeptide in the tumor cell, inhibits or suppresses growth of the cell. Also provided by the present invention is a method suppressing growth of a tumor cell, comprising introducing into the tumor cell a polynucleotide encoding a polypeptide having the amino acid sequence shown in SEQ ID NO: 1 or a fragment thereof, wherein expression of the polypeptide in the tumor cell, suppresses growth of the tumor cell. Nucleic acids encoding functional fragments of SEQ ID NO: 1 can also be utilized in the methods of suppressing tumor cell growth described herein. A functional fragment of the present invention is a fragment of a SKCG-1 polypeptide that still retains at least one activity of SKCG-1, such as tumor suppressive activity or inhibition of cell growth.

The cell can be in vitro, in vivo or ex vivo. Also, the cell can be any cell which can take up and express exogenous nucleic acid and produce a SKCG-1 polypeptide or fragment thereof. The cell of the present invention is not limited to a tumor cell or any other malignant cell as the methods of the present invention can be utilized in vitro, in vivo or ex vivo to deliver SKCG-1 nucleic acids or SKCG-1 polypeptides to any cell for which decreased cell growth is desired. SKCG-1 or a nucleic acid encoding SKCG-1 can be administerd to cells in vitro to determine the extent of cell growth inhibition of a particular cell type prior to ex vivo or in vivo use.

As utilized herein, “suppression or inhibition of cell growth” means a decrease in cell growth which can be, but is not limited to a decrease in cell division, a decrease in the number of cells and/or a decrease in cell size as compared to a control. The control can be a control cell that is not contacted with a polypeptide or nucleic acid of the invention or can be a treated cell before or after contact. This decrease does not have to be complete and can range from a slight decrease in cell growth to a complete elimination or arrest of cell growth.

Delivery of the nucleic acid to cells can be via a variety of mechanisms. As one example, delivery can be via a liposome, using commercially available liposome preparations such as LIPOFECTIN, LIPOFECTAMINE (GIBCO-BRL, Inc., Gaithersburg, Md.), SUPERFECT (Qiagen, Inc. Hilden, Germany) and TRANSFECTAM (Promega Biotec, Inc., Madison, Wis.), as well as other liposomes developed according to procedures standard in the art. In addition, the nucleic acid or vector of this invention can be delivered in vivo by electroporation, the technology for which is available from Genetronics, Inc. (San Diego, Calif.) as well as by means of a SONOPORATION machine (ImaRx Pharmaceutical Corp., Tucson, Ariz.).

As one example, the nucleic acids encoding SKCG-1 polypeptides or fragments thereof, can be delivered in a plasmid or a vector. The polynucleotides encoding SKCG-1 or a fragment thereof can be operably linked to a retroviral long-terminal repeat, a cytomegalovirus promoter, a β-actin promoter, a glucocorticoid-inducible promoter, a SV40 early region promoter or a herpes simplex virus thymidine kinase promoter.

Vector delivery can be via a viral system, such as a retroviral vector system which can package a recombinant retroviral genome (see e.g., Pastan et al., Proc. Natl. Acad. Sci. U.S.A. 85:4486, 1988; Miller et al., Mol. Cell. Biol. 6:2895, 1986). The recombinant retrovirus can then be used to infect and thereby deliver to the infected cells nucleic acid encoding a broadly neutralizing antibody (or active fragment thereof) of the invention. The exact method of introducing the altered nucleic acid into mammalian cells is, of course, not limited to the use of retroviral vectors. Other techniques are widely available for this procedure including the use of adenoviral vectors (Mitani et al., Hum. Gene Ther. 5:941-948, 1994), adeno-associated viral (AAV) vectors (Goodman et al., Blood 84:1492-1500, 1994), lentiviral vectors (Naidini et al., Science 272:263-267, 1996), and pseudotyped retroviral vectors (Agrawal et al., Exper. Hematol. 24:738-747, 1996). Physical transduction techniques can also be used, such as liposome delivery and receptor-mediated and other endocytosis mechanisms (see, for example, Schwartzenberger et al., Blood 87:472-478, 1996) to name a few examples. The liposomes, plasmids and viral vectors utilized for delivery can be conjugated to a targeting moiety that targets specific cell types. For example, the targeting moiety can be a ligand that binds to a tumor specific receptor on a breast tumor cell, or a ligand that binds a tumor specific receptor on a brain tumor cell. The targeting moiety can also be an antibody. Additionally, the plasmids and viral vectors of the present invention can be delivered in a liposome. This invention can be used in conjunction with any of these or other commonly used gene transfer methods.

Also provided by the present invention is a method of suppressing tumor cell growth in a subject, comprising introducing into the tumor cell in the subject, an expression vector comprising a polynucleotide encoding a polypeptide having the amino acid sequence of SEQ ID NO: 1, a variant or a fragment thereof, wherein expression of the polypeptide in the tumor cell in the subject, suppresses growth of the tumor cell in the subject.

A nucleic acid encoding a SKCG-1 polypeptide of this invention can be delivered to the cells of a subject using the mechanisms described above. Dosages can also be determined by one of skill in the art. For example, if the nucleic acid encoding SKCG-1 is delivered to the cells of a subject in an adenovirus vector, the dosage for administration of adenovirus to humans can range from about 10⁷ to 10⁹ plaque forming units (pfu) per injection but can be as high as 10¹² pfu per injection (Crystal, Hum. Gene Ther. 8:985-1001, 1997; Alvarez and Curiel, Hum. Gene Ther. 8:597-613, 1997). A subject can receive a single injection, or, if additional injections are necessary, they can be repeated at six month intervals (or other appropriate time intervals, as determined by the skilled practitioner) for an indefinite period and/or until the efficacy of the treatment has been established.

Parenteral administration of the nucleic acid or vector of the present invention, if used, is generally characterized by injection. Injectables can be prepared in conventional forms, either as liquid solutions or suspensions, solid forms suitable for solution of suspension in liquid prior to injection, or as emulsions. A more recently revised approach for parenteral administration involves use of a slow release or sustained release system such that a constant dosage is maintained. See, e.g., U.S. Pat. No. 3,610,795, which is incorporated by reference herein. For additional discussion of suitable formulations and various routes of administration of therapeutic compounds, see, e.g., Remington: The Science and Practice of Pharmacy (19th ed.) ed. A. R. Gennaro, Mack Publishing Company, Easton, Pa. 1995.

Pharmaceutically Acceptable Carriers

The substances of the present invention, including antibodies, can be used therapeutically in combination with a pharmaceutically acceptable carrier. By pharmaceutically acceptable is meant a material that is not biologically or otherwise undesirable, i.e., the material may be administered to a subject, along with the substance, without causing any undesirable biological effects or interacting in a deleterious manner with any of the other components of the pharmaceutical composition in which it is contained. The carrier would naturally be selected to minimize any degradation of the active ingredient and to minimize any adverse side effects in the subject, as would be well known to one of skill in the art.

Pharmaceutical carriers are known to those skilled in the art. These most typically would be standard carriers for administration of drugs to humans, including solutions such as sterile water, saline, and buffered solutions at physiological pH. The compositions can be administered intramuscularly or subcutaneously. Other compounds will be administered according to standard procedures used by those skilled in the art.

Pharmaceutical compositions may include carriers, thickeners, diluents, buffers, preservatives, surface active agents and the like in addition to the molecule of choice. Pharmaceutical compositions may also include one or more active ingredients such as antimicrobial agents, antiinflammatory agents, anesthetics, and the like.

The pharmaceutical composition may be administered in a number of ways depending on whether local or systemic treatment is desired, and on the area to be treated. Administration may be topically (including rectally, intranasally), orally, by inhalation, or parenterally, for example by intravenous drip, subcutaneous, intraperitoneal or intramuscular injection. The disclosed substances can be administered intravenously, intraperitoneally, intramuscularly, subcutaneously, intracavity, or transdermally.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Formulations for topical administration may include ointments, lotions, creams, gels, drops, suppositories, sprays, liquids and powders. Conventional pharmaceutical carriers, aqueous, powder or oily bases, thickeners and the like may be necessary or desirable.

Compositions for oral administration include powders or granules, suspensions or solutions in water or non-aqueous media, capsules, sachets, or tablets. Thickeners, flavorings, diluents, emulsifiers, dispersing aids or binders may be desirable.

Some of the compositions may potentially be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, trialkyl and aryl amines and substituted ethanolamines.

### EXAMPLES

### Identification of SKCG-1

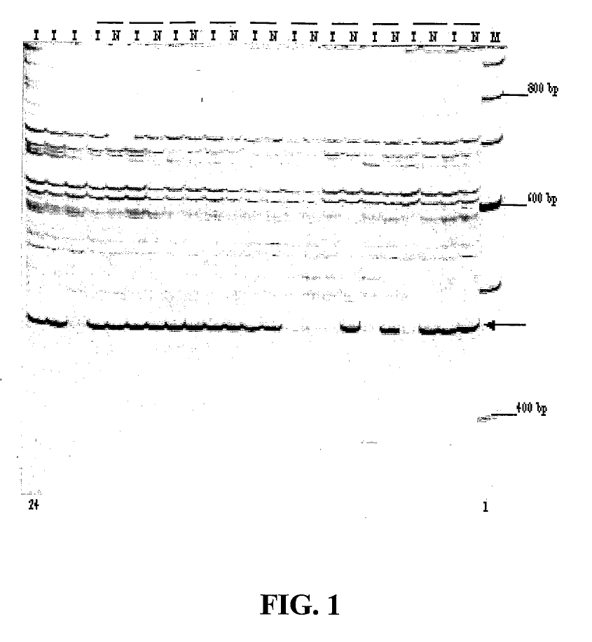

To screen for mutations at a genome-wide level, the genomic fingerprint patterns generated by AP-PCR (1,2) in human Wilms tumors and nonmalignant (uninvolved) tissues from the same subject were compared. Out of 15 different random primers used, one primer (OPA14) revealed multifold decreased intensity of 466 bp AP-PCR amplified DNA fragment in 36% ( 5/14) of the tumor samples as compared to uninvolved kidney tissues from the same individuals (FIG. 1). Out of these five patients, one (a 5 year old Caucasian-American male, FIG. 1, lane 5) was male and the remaining 4 (10 month old African-American female, FIG. 1, lane 7; 5 year old Caucasian-American female, FIG. 1, lane 9; 2 year old African-American female, FIG. 1, lane 11; 4 year old Caucasian-American female, FIG. 1, lane 22) were female. Further characterization of this mutated region revealed that it represents an uncharacterized novel gene that was SKCG-1

SKCG-1 has Point Mutations in Kidney Tumors

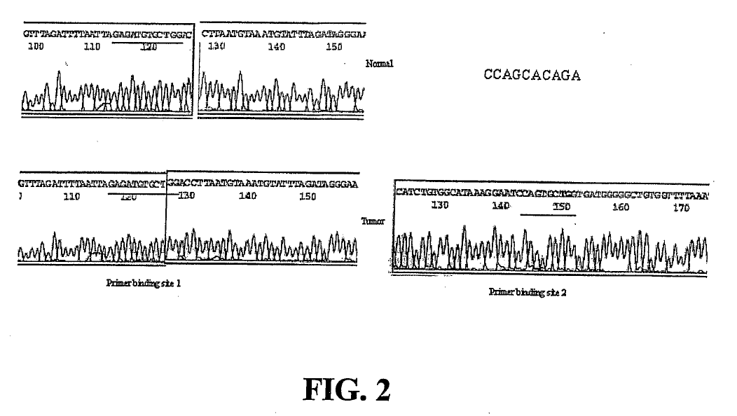

To investigate the mutation at the primer binding site, each of the two priming sites of the 466 bp AP-PCR product were amplified using the primers designed from the flanking region of each priming site. Sequence analysis of these PCR-amplified products from priming sites revealed mutation [CCAGCACAGA (SEQ ID NO: 5) (in normal)→CCAGTGCTGG (SEQ ID NO: 6) (in tumor)] at one of the primer binding sites in the DNA from tumors (FIG. 2).

Full-Length cDNA of SKCG-1 is 2.092 kb, has Characteristic Sequences of a Gene (Translation Start Site, Polyadenylation Signal Sequence) and Encodes an ORF of 124 Amino Acids

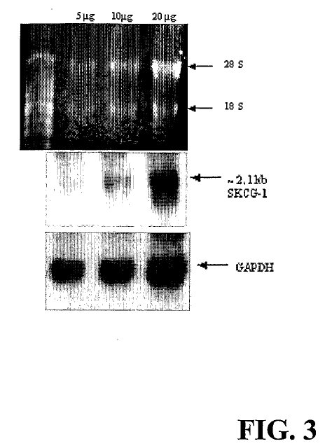

Northern analysis on normal human kidney epithelial cells (HK-2) using a PCR generated probe from 466 bp AP-PCR region revealed a transcript of 2.1 kb (FIG. 3). A sequence homology search revealed 100% similarity of the 466 bp AP-PCR fragment sequence with an EST clone. A full-length cDNA sequence of SKCG-1 was isolated by sequencing this EST clone and further conducting 5′ and 3′RACE on it.

Sequence analysis of the 2.092 kb full-length cDNA and 1 kb upstream of the 5′ end of this transcript revealed a promoter region sequence (TATA box) at 13 bp upstream of the 5′ end of the cDNA, a translation start site (ATG sequence) present after 110 bp of the transcription start site and a polyadenylation signal sequence (AATAAA) (SEQ ID NO: 7), present at 80 bp upstream of 3′ end of cDNA sequence. ORF analysis revealed that the cDNA sequence of this gene has an open reading frame encoding a protein of 124 amino acids in frame +2. Domain analysis revealed that this gene does not have any conserved functional domain. No significant homology of this gene with any known gene was found.

SKCG-1 Gene Transcript is Present in Various Human Normal Tissues and Absent in Wilms, RCC and Breast Tumor Tissues

To determine the distribution of SKCG-1 gene transcript in various human tissues PCR amplification with SKCG-1 gene specific primer was performed on a panel of cDNAs from various human tissues (Origene Inc). A SKCG-1 gene specific amplification product of expected size was abundantly present in brain, kidney, liver, testis, adrenal gland, fetal brain, fetal liver whereas relatively less expression of this gene transcript was found in heart, small intestine, muscle, stomach, placenta, and prostate. No expression of this gene transcript was detected in spleen, colon, lung, salivary gland, pancreas, uterus, skin, PBL, and bone marrow.

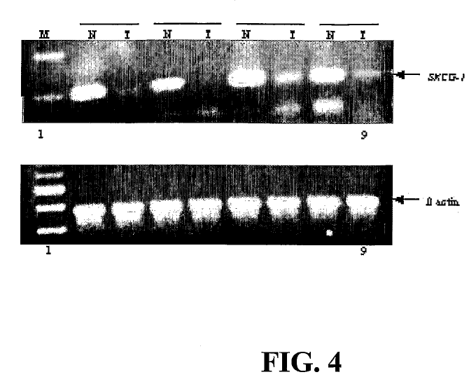

Using 2 sets of primer combinations, the expression pattern of SKCG-1 gene transcript was analyzed by RT-PCR analysis in 5 Wilms, 2 RCC and 2 Breast tumor samples versus corresponding uninvolved tissues. Expression of this gene was found either completely absent or hardly detectable in five Wilms tumors (those samples showing loss of the 475 bp AP-PCR locus), and two renal rell rarcinomas and two breast tumors, however its expression was readily detectable in their corresponding uninvolved tissues (FIG. 4). Determination of SKCG-1 gene transcript level by RNase protection assay with a few samples also confirmed the absence of SKCG-1 transcript in Wilms tumors.

SKCG-1 Gene is Located on q23.2 Region of Chromosome 11, a Highly Unstable Chromosome in Human Cancers

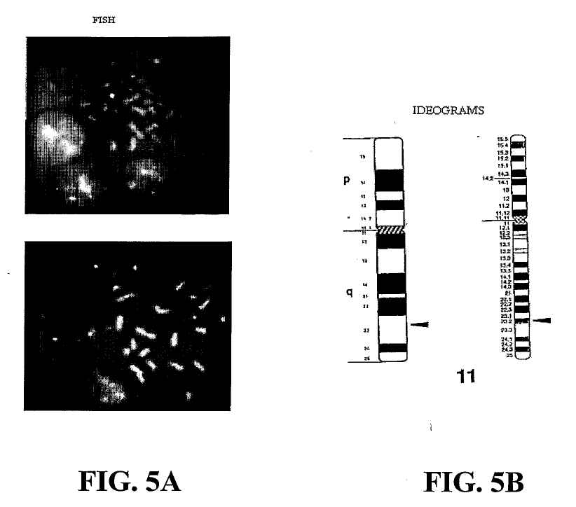

An initial FISH experiment with a human BAC clone containing the 466 bp region of the SKCG-1 gene as a probe resulted in the specific labeling of the long arm of a group C chromosome that was believed to be chromosome 11 on the basis of size, morphology, and banding pattern. A second experiment with a biotin labeled probe, which was specific for the centromere of chromosome 11, was cohybridized with SKCG-1 BAC clone. This experiment resulted in the specific labeling of the centromere (red) and the long arm (green) of chromosome 11. Measurement of 10 specifically labeled chromosome 11 demonstrated that the SKCG-1 BAC clone is located at a position, which is 73% of the distance from the centromere to the telomere of chromosome arm 11q, an area that corresponds to band 11q23.2 (FIG. 5). A total of 80 metaphase cells were analyzed with 75 exhibiting specific labeling.

Serum-Stress (Starvation) Induces SKCG-1 Gene Transcript in Kidney Epithelial Cells

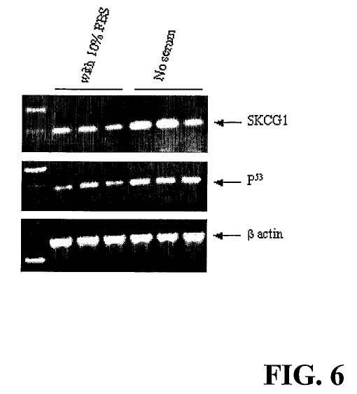

Expression of SKCG-1 gene transcript was analyzed by RT-PCR using the RNA isolated from HEK-293 embryonic kidney epithelial cells grown with 10% FBS or without serum for 48 and 72 hrs. Gene expression data revealed that kidney epithelial cells grown without serum (apoptotic cells) had multifold increases in transcript levels as compared to the actively growing kidney epithelial cells grown with serum for the same time period (FIG. 6).

SKCG-1 Controls the Growth/Proliferation of Kidney Epithelial Cells

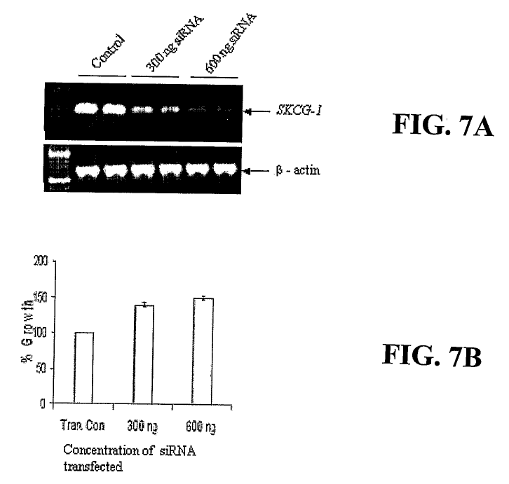

To determine the extent of gene silencing by siRNA, after 48 hrs of transfection total RNA was isolated and semiquantitative RT-PCR was performed. The SKCG-1 gene transcript in siRNA-transfected cells was much less as compared to controls, indicating that the gene is significantly silenced (FIG. 7a). To investigate whether the silencing of SKCG-1 gene has any effect on the control of cell growth, cells counts were performed prior to isolating the RNA. Comparison of cell count data revealed a 40% increase in the growth of human embryonic kidney cells as a result of SKCG-1 gene silencing as compared to the growth of control cells with constitutive expression of SKCG-1 (FIG. 7b). These data indicate that SKCG-1 plays an important role in the control of growth/proliferation of kidney epithelial cells.

Throughout this application, various publications are referenced. The disclosures of these publications in their entireties are hereby incorporated by reference into this application in order to more fully describe the state of the art to which this invention pertains.

It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

### REFERENCES

1. Singh K P., Roy D. Detection of mutation(s) or polymorphic loci in the genome of experimental animal and human cancer tissues by RAPD/AP-PCR depend on DNA polymerase. [Journal Article] International Journal of Oncology. 14(4):753-8, 1999
2. Singh K P., Roy D. Identification of novel breast tumor-specific mutation(s) in the q11.2 region of chromosome 17 by RAPD/AP-PCR fingerprinting. Gene. 269(1-2):33-43, 2001
3. Bernadette Olives, Sonia Martial, Marie-Genevieve Maftei, Giorgio Matassi, Germain Rousselet, Pierre Ripoche, Jean-Pierre Cartron and Pascal Bailly 1996. Molecular characterization of a new urea transporter in the human kidney. FEBS Lett. 386 (2-3), 156-160.

What is claimed is:

1. A purified polypeptide comprising SEQ ID NO: 1.

2. A purified polypeptide comprising an amino acid sequence at least about 95% identical to the sequence of SEQ ID NO: 1.

3. A purified polypeptide comprising the amino acid sequence of SEQ ID NO: 1 with one or more conservative amino acid substitutions.

4. A nucleic acid encoding the polypeptide of claim 1.

5. The nucleic acid of claim 4, wherein the nucleic acid comprises SEQ ID NO: 2.

6. A vector comprising the nucleic acid of claim 4 or 5.

7. A host cell comprising the vector of claim 6.

8. The host cell of claim 7, wherein the host cell is a prokaryotic cell.

9. The host cell of claim 7, wherein the host cell is a eukaryotic cell.

10. An isolated antibody or fragment thereof that specifically binds the polypeptide of claim 1.

11. The antibody of claim 10, wherein the antibody is a polyclonal antibody.

12. The antibody of claim 10, wherein the antibody is a monoclonal antibody.

13. The antibody of claim 10, wherein the antibody is labeled with a detectable moiety.

14. The antibody of claim 13, wherein the detectable moiety is selected from the group consisting of a fluorescent moiety, an enzyme-linked moiety, a biotinylated moiety and a radiolabeled moiety.

15. The antibody of claim 10, wherein the antibody is humanized.

16. A method for detecting expression of a tumor suppressor gene in a subject, comprising analyzing a sample from the subject for expression of a SKCG-1 gene.

17. The method of claim 16, wherein the SKCG-1 gene comprises the nucleic acid sequence of SEQ ID NO: 2.

18. The method of claim 16, wherein reduced expression of the SKCG-1 gene indicates the subject has or is at risk for developing cancer.

19. A method for determining whether a subject has or is at risk for developing cancer comprising: detecting reduced expression of the SKCG-1 gene in a test sample from the subject as compared to a control sample, wherein reduced expression of the SKCG-1 gene in the test sample indicates that the subject has or is at risk for developing cancer.

20. A method for determining whether a subject has or is at risk for developing cancer comprising: analyzing the methylation status of a SKCG-1 gene in a sample from the subject and detecting a difference in methylation status as compared to that of a comparable normal cell, wherein the difference in methylation status indicates that the subject has or is at risk of developing cancer.

21. A method for determining whether a subject has or is at risk for developing cancer comprising: detecting the presence or absence in the SKCG-1 gene of said subject a genetic polymorphism comprising the nucleotide sequence set forth in SEQ ID NO:3, with one or more point mutations, wherein the point mutations comprise point mutations located between nucleotide position 1546 to nucleotide position 1555, and wherein the presence of the genetic polymorphism indicates that the subject has or is at risk of developing cancer.

22. The method of any one of claims 19-21, wherein the cancer is selected from the group consisting of breast cancer, renal cancer and ovarian cancer.

23. A method of suppressing growth of a tumor cell, comprising introducing into the tumor cell an expression vector comprising a polynucleotide encoding a polypeptide having the amino acid sequence shown in SEQ ID NO: 1, wherein expression of the polypeptide in the tumor cell, suppresses growth of the tumor cell.

24. The method of claim 23 in which the cell is a human tumor cell.

25. The method of claim 24 in which the tumor cell is a breast carcinoma cell, a renal carcinoma cell or an ovarian carcinoma cell;

26. The method of claim 23 in which the expression vector is a viral vector.

27. The method of claim 26 in which the viral vector is a retroviral vector or an adenoviral vector.

28. The method of claim 23 in which the expression vector is a plasmid.

29. The method of claim 23 in which the polynucleotide is operably linked to a retroviral long-terminal repeat, a cytomegalovirus promoter, a β-actin promoter, a glucocorticoid-inducible promoter, a SV40 early region promoter or a herpes simplex virus thymidine kinase promoter.

30. The method of claim 23 in which the expression vector is introduced into the cell in vitro.

31. The method of claim 23 in which the expression vector is introduced into the cell in vivo.

32. A method of suppressing tumor cell growth in a subject, comprising introducing into the tumor cell in the subject, an expression vector comprising a polynucleotide encoding a polypeptide having the amino acid sequence of SEQ ID NO:1, wherein expression of the polypeptide in the tumor cell in the subject, suppresses growth of the tumor cell in the subject.

33. The method of claim 32 in which the cell is a human tumor cell.

34. The method of claim 33 in which the tumor cell is a breast carcinoma cell, a renal carcinoma cell or an ovarian carcinoma cell.

35. The method of claim 32 in which the expression vector is a viral vector.

36. The method of claim 35 in which the viral vector is a retroviral vector or an adenoviral vector.

37. The method of claim 32 in which the expression vector is a plasmid.

38. The method of claim 32 in which the polynucleotide is operably linked to a retroviral long-terminal repeat, a cytomegalovirus promoter, a β-actin promoter, a glucocorticoid-inducible promoter, a SV40 early region promoter or a herpes simplex virus thymidine kinase promoter.

39. The method of claim 32 in which the expression vector is introduced into the cell in vitro.

40. The method of claim 32 in which the expression vector is introduced into the cell in vivo.

41. The method of claim 32 in which the expression vector is delivered by direct injection.
